(19) United States
(12) Patent Application Publication
(10) Pub. No.: US 2024/0254098 A1
(43) Pub. Date: Aug. 1, 2024

(54) THIOESTER PRODRUGS FOR THE TREATMENT OF RENAL ANOMALIES

(71) Applicant: ALTIBIO, INC., Half Moon Bay, CA (US)

(72) Inventors: Charles M. COOK, Southlake, TX (US); Marc Patrick REICHENBERGER, Half Moon Bay, CA (US); Shripad BHAGWAT, Half Moon Bay, CA (US)

(21) Appl. No.: 18/560,078

(22) PCT Filed: May 10, 2022
(86) PCT No.: PCT/US2022/028642

§ 371 Date: Nov. 9, 2023

Related U.S. Application Data
(60) Provisional application No. 63/186,707, filed on May 10, 2021.

(51) Int. Cl. C07D 333/38; A61K 31/381

(52) U.S. Cl. CPC C07D 333/38; A61K 31/381

(57) ABSTRACT

Provided herein are compounds, thioester prodrugs thereof, compositions, and methods for the treatment of diseases and disorders associated with renal anomalies.

### FIELD

Provided herein are novel thioester prodrugs, and methods for treating a variety of diseases, disorders, and conditions including administration of the thioester prodrugs.

### BACKGROUND

Cystinuria is a rare genetic disease with increased urinary excretion of the poorly soluble cystine, which is the oxidized/dimerized form of the amino acid cysteine. The excretion of this insoluble cystine can lead to significant morbidity in affected patients due to the typically large and recurrent resulting kidney stones. The consequences of cystine stone formation are of such a serious nature as to markedly shorten life expectancy and induce both physical and mental anguish in involved persons.

Current treatments are focused on the prevention of stone formation. Preventative treatments (i.e., mitigation or prevention of cystine stones) is primarily focused on the reduction of the disulfide bond in the insoluble cystine via a thiol drug, which reacts with the cystine to form cysteine and a mixed disulfide, both of which are significantly more soluble than cystine. However, the presence of a thiol (mercaptan) group is known to (1) cause adverse events, especially gastrointestinal (GI) adverse events via oral dosing: (2) have highly variable bioavailability and delayed intestinal absorption; and (3) have a propensity for oxidation over time to inactive disulfides. These effects pervade any drug with the thiol group (i.e., a class effect).

Development of thioester prodrugs should circumvent the aforementioned mercaptan shortcomings by masking the mercaptan in the form of a thioester, thus improving oral bioavailability, increasing intestinal absorption, and reducing mercaptan oxidation to inactive disulfides. Therefore, there is a need for effective treatments of cystinuria, and other related renal anomalies, via thioester prodrugs.

### SUMMARY

Provided herein are compounds useful, for example, in renal treatments.

In one embodiment, provided are compounds of the following formula

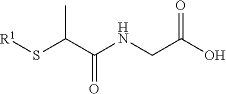

wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl;
wherein alkyl, alkenyl, alkynyl, cycloalkyl, aryl, and heteroaryl are optionally substituted and/or comprise one or more stereogenic centers;
wherein R¹ is not methyl, CH₃(CH₂)₁₆—, CH₃(CH₂)₁₄—, CH₃(CH₂)₁₂—, or CH₃(CH₂)₁₀—,

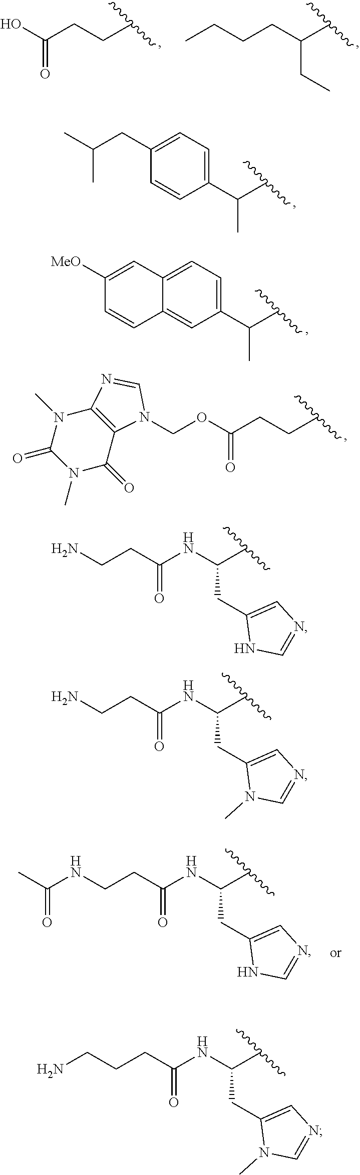

wherein R¹ is not phenyl, 2-acetoxyphenyl, 4-nitrophenyl, or 3,4,5-trimethoxyphenyl;
wherein R¹ is not 3-pyridyl;
wherein R¹ is not

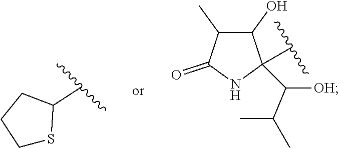

wherein R¹ is not

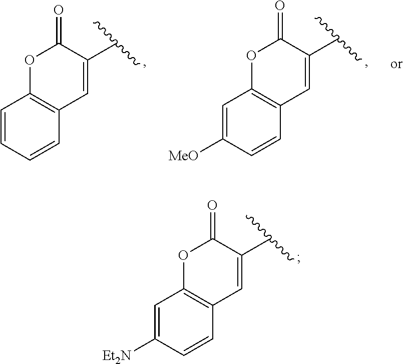

and
wherein when R¹ is

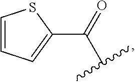 [SMILES: *C(=O)c1cccs1]

then the compound has an ee greater than zero; or
a pharmaceutically acceptable salt thereof.

In another embodiment, provided are compounds of the following formula

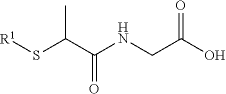

wherein R¹ is —(C═O)alkyl, —(C=O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl:
wherein alkyl, alkenyl, alkynyl, cycloalkyl, aryl, and heteroaryl are optionally substituted:
wherein the compound has an ee greater than zero; or
a pharmaceutically acceptable salt thereof.

In another embodiment, provided is a method for treating a condition, disease, or disorder in a patient comprising administering to the patient a therapeutically effective amount of a compound of the following formula

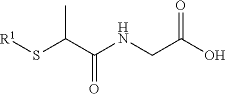

wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl;
wherein alkyl, alkenyl, alkynyl, cycloalkyl, aryl, and heteroaryl are optionally substituted and/or comprise one or more stereogenic centers; and
wherein when R¹ is

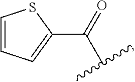 [SMILES: *C(=O)c1cccs1]

then the compound has an ee greater than zero; or
a pharmaceutically acceptable salt thereof.

In another embodiment, provided is a method for treating a kidney disease, a renal disease, a bladder disease, a urinary disease, a ureteral disease, kidney stones, staghorn stones, cystine stones, calculi, lithiasis, nephrolithiasis, urolithiasis, urothiasis, ureterolithiasis, renolithiasis, SLC3A1 gene mutations, SLC7A9 gene mutations, defects in the dibasic amino acid transporter, and cystinuria in a patient comprising administering to the patient a therapeutically effective amount of a compound of the following formula having an ee greater than zero

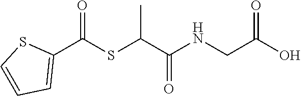 [SMILES: CC(SC(=O)c1cccs1)C(=O)NCC(=O)O]

or a pharmaceutically acceptable salt thereof.

### DESCRIPTION OF EXEMPLARY EMBODIMENTS

Provided herein are compounds, compositions, and methods useful for treating, for example, kidney and/or renal anomalies.

### Definitions

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term provided herein, these Definitions prevail unless stated otherwise.

As used herein, “alkyl” refers to a monovalent and saturated hydrocarbon radical moiety. Alkyl is optionally substituted and can be linear, branched, or cyclic, i.e., cycloalkyl. Alkyl includes, but is not limited to, those radicals having 1-20 carbon atoms, i.e., C₁₋₂₀ alkyl; 1-12 carbon atoms, i.e., C₁₋₁₂ alkyl: 1-8 carbon atoms, i.e., C₁₋₈ alkyl; 1-6 carbon atoms, i.e., C₁₋₆ alkyl; and 1-3 carbon atoms, i.e., C₁₋₃ alkyl. Examples of alkyl moieties include, but are not limited to, methyl, ethyl, n-propyl, i-propyl, n-butyl, s-butyl, t-butyl, i-butyl, a pentyl moiety, a hexyl moiety, cyclopropyl, cyclobutyl, cyclopentyl, and cyclohexyl. A pentyl moiety includes, but is not limited to, n-pentyl and i-pentyl. A hexyl moiety includes, but is not limited to, n-hexyl.

As used herein, “alkylene” refers to a divalent alkyl group. Unless specified otherwise, alkylene includes, but is not limited to, 1-20 carbon atoms. The alkylene group is optionally substitued as described herein for alkyl. In some embodiments, alkylene is unsubstituted.

Designation of an amino acid or amino acid residue without specifying its stereochemistry is intended to encompass the L- form of the amino acid, the D- form of the amino acid, or a racemic mixture thereof.

As used herein, “haloalkyl” refers to alkyl, as defined above, wherein the alkyl includes at least one substituent selected from a halogen, for example, fluorine (F), chlorine (Cl), bromine (Br), or iodine (I). Examples of haloalkyl include, but are not limited to, —CF₃, —CH₂CF₃, —CCl₂F, and —CCl₃.

As used herein, “alkenyl” refers to a monovalent hydrocarbon radical moiety containing at least two carbon atoms and one or more non-aromatic carbon-carbon double bonds. Alkenyl is optionally substituted and can be linear, branched, or cyclic. Alkenyl includes, but is not limited to, those radicals having 2-20 carbon atoms, i.e., C₂₋₂₀ alkenyl; 2-12 carbon atoms, i.e., C₂₋₁₂ alkenyl; 2-8 carbon atoms, i.e., C₂₋₈ alkenyl; 2-6 carbon atoms, i.e., C₂₋₆ alkenyl; and 2-4 carbon atoms, i.e., C₂₋₄ alkenyl. Examples of alkenyl moieties include, but are not limited to, vinyl, propenyl, butenyl, and cyclohexenyl.

As used herein, “alkenylene” refers to a divalent alkenyl group. Unless specified otherwise, alkenylene includes, but is not limited to, 2-20 carbon atoms. The alkenylene group is optionally substitued as described herein for alkyl. In some embodiments, alkenylene is unsubstituted.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

As used herein, “alkynylene” refers to a divalent alkynyl group. Unless specified otherwise, alkynylene includes, but is not limited to, 2-20 carbon atoms. The alkynylene group is optionally substitued as described herein for alkyl. In some embodiments, alkynylene is unsubstituted.

As used herein, “alkoxy” refers to a monovalent and saturated hydrocarbon radical moiety wherein the hydrocarbon includes a single bond to an oxygen atom and wherein the radical is localized on the oxygen atom, e.g., CH₃CH₂—O· for ethoxy. Alkoxy substituents bond to the compound which they substitute through this oxygen atom of the alkoxy substituent. Alkoxy is optionally substituted and can be linear, branched, or cyclic, i.e., cycloalkoxy. Alkoxy includes, but is not limited to, those having 1-20 carbon atoms, i.e., C₁₋₂₀ alkoxy: 1-12 carbon atoms, i.e., C₁₋₁₂ alkoxy: 1-8 carbon atoms, i.e., C₁₋₈ alkoxy: 1-6 carbon atoms, i.e., C₁₋₆ alkoxy; and 1-3 carbon atoms, i.e., C₁₋₃ alkoxy. Examples of alkoxy moieties include, but are not limited to, methoxy, ethoxy, n-propoxy, i-propoxy, n-butoxy, s-butoxy, t-butoxy, i-butoxy, a pentoxy moiety, a hexoxy moiety, cyclopropoxy, cyclobutoxy, cyclopentoxy, and cyclohexoxy.

As used herein, “haloalkoxy” refers to alkoxy, as defined above, wherein the alkoxy includes at least one substituent selected from a halogen, e.g., F, Cl, Br, or I.

As used herein, “alkoxyalkyl” refers to alkyl, as defined above, wherein the alkyl includes at least one alkoxy substituent, as defined above.

As used herein, “aryl” refers to a monovalent moiety that is a radical of an aromatic compound wherein the ring atoms are carbon atoms. Aryl is optionally substituted and can be monocyclic or polycyclic, e.g., bicyclic or tricyclic. Examples of aryl moieties include, but are not limited to, those having 6 to 20 ring carbon atoms, i.e., C₆₋₂₀ aryl; 6 to 15 ring carbon atoms, i.e., C₆₋₁₅ aryl, and 6 to 10 ring carbon atoms, i.e., C₆₋₁₀ aryl. Examples of aryl moieties include, but are limited to, phenyl, naphthyl, fluorenyl, azulenyl, anthryl, phenanthryl, and pyrenyl.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g.,

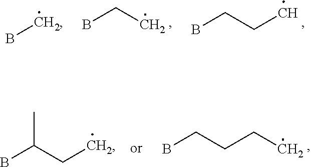

wherein B is an aromatic moiety, e.g., aryl or phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g.,

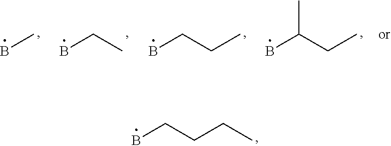

wherein B is an aromatic moiety, e.g., phenyl. Alkylaryl is optionally substituted, i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g.,

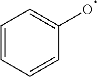

for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C_{6- 15} aryloxy, and 6 to 10 ring carbon atoms, i.e., C₆₋₁₀ aryloxy. Examples of aryloxy moieties include, but are not limited to phenoxy, naphthoxy, and anthroxy.

As used herein, “arylene” refers to a divalent moiety of an aromatic compound wherein the ring atoms are only carbon atoms. Arylene is optionally substituted and can be monocyclic or polycyclic, e.g., bicyclic or tricyclic. Examples of arylene moieties include, but are not limited to those having 6 to 20 ring carbon atoms, i.e., C₆₋₂₀ arylene; 6 to 15 ring carbon atoms, i.e., C6-15 arylene, and 6 to 10 ring carbon atoms, i.e., C₆₋₁₀ arylene.

As used herein, “heteroalkyl” refers to an alkyl in which one or more carbon atoms are replaced by heteroatoms. As used herein, “heteroalkenyl” refers to an alkenyl in which one or more carbon atoms are replaced by heteroatoms. As used herein, “heteroalkynyl” refers to an alkynyl in which one or more carbon atoms are replaced by heteroatoms. Suitable heteroatoms include, but are not limited to, nitrogen, oxygen, and sulfur atoms. Heteroalkyl, heteroalkenyl, and heteroalkynyl are optionally substituted. Examples of heteroalkyl moieties include, but are not limited to, aminoalkyl, sulfonylalkyl, and sulfinylalkyl. Examples of heteroalkyl moieties also include, but are not limited to, methylamino, methylsulfonyl, and methylsulfinyl.

As used herein, “heteroaryl” refers to a monovalent moiety that is a radical of an aromatic compound wherein the ring atoms contain carbon atoms and at least one oxygen, sulfur, nitrogen, or phosphorus atom. Examples of heteroaryl moieties include, but are not limited to those having 5 to 20 ring atoms; 5 to 15 ring atoms; and 5 to 10 ring atoms. Heteroaryl is optionally substituted.

As used herein, “heteroarylene” refers to a divalent heteroaryl in which one or more ring atoms of the aromatic ring are replaced with an oxygen, sulfur, nitrogen, or phosphorus atom. Heteroarylene is optionally substituted.

As used herein, “heterocycloalkyl” refers to a cycloalkyl in which one or more carbon atoms are replaced by heteroatoms. Suitable heteroatoms include, but are not limited to, nitrogen, oxygen, and sulfur atoms. Heterocycloalkyl is optionally substituted. Examples of heterocycloalkyl moieties include, but are not limited to, tetrahydropyranyl, dioxolanyl, pyrrolidinyl, piperidinyl, morpholinyl, thianyl, and dithiolanyl.

As used herein, “heterocyclyl” refers to an unsaturated or nonaromatic cycloalkyl in which one or more carbons atoms are replaced by heteroatoms. Suitable heteroatoms include, but are not limited to, nitrogen, oxygen, and sulfur atoms. Heterocyclyl is optionally substituted. Examples of heterocyclyl moieties include 2H-pyrrole, 2H-pyranyl, and coumarinyl.

As used herein, “Lewis acid” refers to a molecule or ion that accepts an electron lone pair. The Lewis acids used in the methods described herein are those other than protons. Lewis acids include, but are not limited to, non-metal acids, metal acids, hard Lewis acids, and soft Lewis acids. Lewis acids include, but are not limited to, Lewis acids of aluminum, boron, iron, tin, titanium, magnesium, copper, antimony, phosphorus, silver, ytterbium, scandium, nickel, and zinc. Illustrative Lewis acids include, but are not limited to, AlBr₃, AlCl₃, BCl₃, boron trichloride methyl sulfide, BF₃, boron trifluoride methyl etherate, boron trifluoride methyl sulfide, boron trifluoride tetrahydrofuran, dicyclohexylboron trifluoromethanesulfonate, iron (III) bromide, iron (III) chloride, tin (IV) chloride, titanium (IV) chloride, titanium (IV) isopropoxide, Cu(OTf)₂, CuCl₂, CuBr₂, zinc chloride, alkylaluminum halides (R₃₋ₙAlXₙ, wherein R is hydrocarbyl; X is a halogen, such as chlorine; and n is 1-3), Zn(OTf)₂, ZnCl₂, Yb(OTf)₃, Sc(OTf)₃, MgBr₂, NiCl₂, Sn(OTf)₂, Ni(OTf)₂, and Mg(OTf)₂.

As used herein, “N-containing heterocycloalkyl,” refers to a cycloalkyl in which one or more carbon atoms are replaced by heteroatoms and wherein at least one replacing heteroatom is a nitrogen atom. Suitable heteroatoms in addition to nitrogen, include, but are not limited to, oxygen and sulfur atoms. N-containing heterocycloalkyl is optionally substituted. Examples of N-containing heterocycloalkyl moieties include, but are not limited to, morpholinyl, piperidinyl, pyrrolidinyl, imidazolidinyl, oxazolidinyl, or thiazolidinyl.

As used herein, “optionally substituted,” when used to describe a radical moiety, for example, optionally substituted alkyl, means that such moiety is optionally bonded to one or more substituents. Examples of such substituents include, but are not limited to, halo, cyano, nitro, amino, hydroxyl, optionally substituted haloalkyl, aminoalkyl, hydroxyalkyl, azido, epoxy, optionally substituted heteroaryl, optionally substituted heterocycloalkyl,

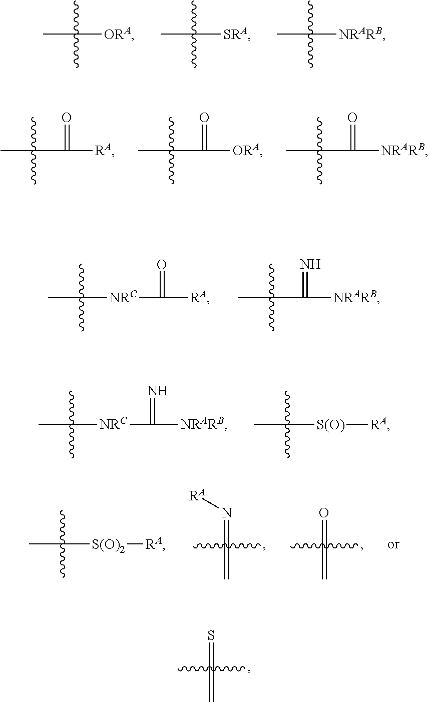

wherein R^{A}, R^{B}, and R^{C} are, independently at each occurrence, a hydrogen atom, alkyl, alkenyl, alkynyl, aryl, alkylaryl, arylalkyl, heteroalkyl, heteroaryl, or heterocycloalkyl, or R^{A} and R^{B} together with the atoms to which they are bonded, form a saturated or unsaturated carbocyclic ring, wherein the ring is optionally substituted, and wherein one or more ring atoms is optionally replaced with a heteroatom. In certain embodiments, when a radical moiety is optionally substituted with an optionally substituted heteroaryl, optionally substituted heterocycloalkyl, or optionally substituted saturated or unsaturated carbocyclic ring, the substituents on the optionally substituted heteroaryl, optionally substituted heterocycloalkyl, or optionally substituted saturated or unsaturated carbocyclic ring, if they are substituted, are not substituted with substituents which are further optionally substituted with additional substituents. In some embodiments, when a group described herein is optionally substituted, the substituent bonded to the group is unsubstituted unless otherwise specified.

As used herein, “amide synthesis conditions” refers to reaction conditions suitable to effect the formation of an amide, e.g., by the reaction of a carboxylic acid, activated carboxylic acid, or acyl halide with an amine. In some examples, amide synthesis conditions refers to reaction conditions suitable to effect the formation of an amide bond between a carboxylic acid and an amine. In some of these examples, the carboxylic acid is first converted to an activated carboxylic acid before the activated carboxylic acid reacts with an amine to form an amide. Suitable conditions to effect the formation of an amide include, but are not limited to, those utilizing reagents to effect the reaction between a carboxylic acid and an amine, including, but not limited to, dicyclohexylcarbodiimide (DCC), diisopropylcarbodiimide (DIC), (benzotriazol-1-yloxy)tris(dimethylamino)phosphonium hexafluorophosphate (BOP), (benzotriazol-1-yloxy)tripyrrolidinophosphonium hexafluorophosphate (PyBOP), (7-azabenzotriazol-1-yloxy)tripyrrolidinophosphonium hexafluorophosphate (PyAOP), bromotripyrrolidinophosphonium hexafluorophosphate (PyBrOP), O-(benzotriazol-1-yl)-N,N,N′,N′-tetramethyluronium hexafluorophosphate (HBTU), O-(benzotriazol-1-yl)-N,N,N′,N′-tetramethyluronium tetrafluoroborate (TBTU), 1-[Bis(dimethylamino)methylene]-1H-1,2,3-triazolo[4,5-b]pyridinium 3-oxide hexafluorophosphate (HATU), N-ethoxycarbonyl-2-ethoxy-1,2-dihydroquinoline (EEDQ), N-ethyl-N′-(3-dimethylaminopropyl)carbodiimide (EDC), 2-chloro-1,3-dimethylimidazolidinium hexafluorophosphate (CIP), 2-chloro-4,6-dimethoxy-1,3,5-triazine (CDMT), and carbonyldiimidazole (CDI). In some examples, a carboxylic acid is first converted to an activated carboxylic ester before treating the activated carboxylic ester with an amine to form an amide bond. In certain embodiments, the carboxylic acid is treated with a reagent. The reagent activates the carboxylic acid by deprotonating the carboxylic acid and then forming a product complex with the deprotonated carboxylic acid as a result of nucleophilic attack by the deprotonated carboxylic acid onto the protonated reagent. The activated carboxylic esters for certain carboxylic acids are subsequently more susceptible to nucleophilic attack by an amine than the carboxylic acid is before it is activated. This results in amide bond formation. As such, the carboxylic acid is described as activated. Exemplary reagents include DCC and DIC.

As used herein, “stereogenic center” refers to isomeric molecules that include atoms which have the same connectivity yet differ in the spatial arrangement of the atoms. As would be appreciated by a person of skill, stereogenic centers can be assigned as (R)- or (S)- according to the Cahn, Ingold, Prelog rules. Stereogenic centers include, without limitation, carbon atoms, phosphorous atoms, quaternary nitrogen atoms, and sulfur atoms.

As used herein, “enantiomeric excess (ee)” refers to a dimensionless mole ratio describing the purity of chiral substances that contain, for example, a single stereogenic center. For instance, an enantiomeric excess of zero would indicate a racemic (e.g., 50:50 mixture of enantiomers, or no excess of one enantiomer over the other). By way of further example, an enantiomeric excess of ninety-nine would indicate a nearly stereopure enantiomeric compound (i.e., large excess of one enantiomer over the other). The percentage enantiomeric excess, % ee=([(R)-compound]−[(S)-compound])/([(R)-compound]+[(S)-compound])×100, where the (R)-compound>(S)-compound; or % ee=([(S)-compound]−[(R)-compound])/([(S)-compound]+[(R)-compound])×100, where the (S)-compound>(R)-compound.

As used herein, “diastereomeric excess (de)” refers to a dimensionless mole ratio describing the purity of chiral substances that contain more than one stereogenic center. For example, a diastereomeric excess of zero would indicate an equimolar mixture of diastereoisomers. By way of further example, diastereomeric excess of ninety-nine would indicate a nearly stereopure diastereomeric compound (i.e., large excess of one diastereomer over the other). Diastereomeric excess may be calculated via a similar method to ee. As would be appreciated by a person of skill, de is usually reported as percent de (% de). % de may be calculated in a similar manner to % ee.

As used herein, the term “residue” refers to the chemical moiety within a compound that remains after a chemical reaction. For example, the phrase “amino acid residue” or “thioester residue” refers to the product of an amide coupling or peptide coupling of an amino acid, or the product of an esterification of a suitable coupling partner (e.g., a mercaptan), respectively; wherein, for example, a water molecule is expelled after the amide or peptide coupling of the amino acid or the thioester coupling, resulting in the product having the amino acid residue or thioester residue incorporated therein.

As used herein, “therapeutically effective amount” refers to an amount (e.g., of a compound) that is sufficient to provide a therapeutic benefit to a patient in the treatment or management of a disease or disorder, or to delay or minimize one or more symptoms associated with the disease or disorder.

As used herein, “constitutional isomers” refers to compounds that have the same molecular formula, but different chemical structures resulting from the way the atoms are arranged. Exemplary constitutional isomers include n-propyl and isopropyl; n-butyl, sec-butyl, and tert-butyl; and n-pentyl, isopentyl, and neopentyl, and the like.

Certain groups, moieties, substituents, and atoms are depicted with a wiggly line that intersects a bond or bonds to indicate the atom through which the groups, moieties, substituents, or atoms are bonded. For example, a phenyl group that is substituted with a propyl group depicted as:

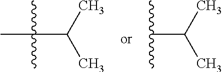

has the following structure:

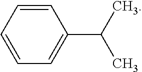

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains. For example, the group,

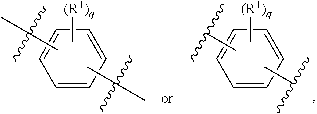

wherein subscript q is an integer from 0 to 4 and in which the positions of substituent R¹ are described generically, i.e., not directly attached to any vertex of the bond line structure, i.e., specific ring carbon atom, includes the following, non-limiting examples of groups in which the substituent R¹ is bonded to a specific ring carbon atom:

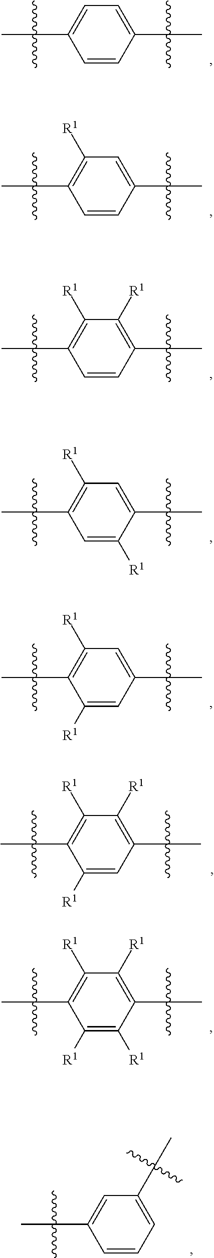

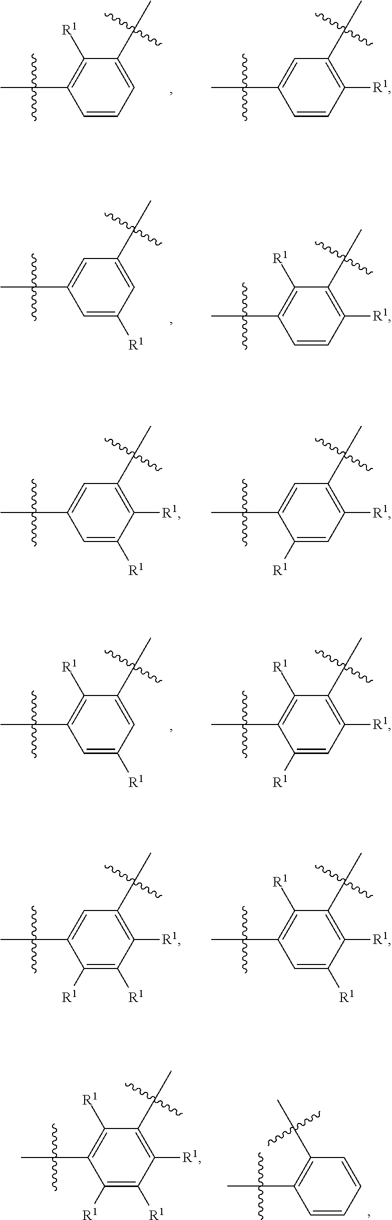

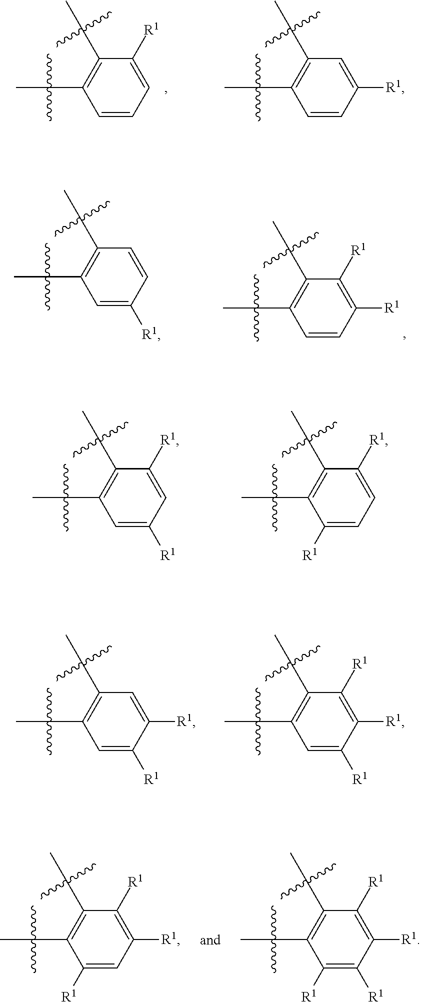

### Compounds or Thioesters

Provided herein are compounds or thioesters. Without being bound by any particular theory of operation, the compounds include thioester derivatives. In certain embodiments, the compounds are capable of carrying out any activity of thioesters or a thioester derivative at or in a target, for instance, a target cell. Certain compounds can have one or more additional activities. In certain embodiments, the compounds are capable of modulating the activity of a transporter (e.g., a dibasic amino acid transporter). The following embodiments of compounds or thioesters are contemplated.

In one embodiment, the compound is

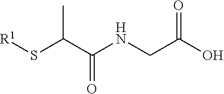

wherein R¹ is —(C═O)alkyl, wherein alkyl is optionally substituted and/or comprises one or more stereogenic centers. In one embodiment, R¹ is —(C═O)alkyl, wherein alkyl is optionally substituted and/or comprises one or more stereogenic centers, and wherein alkyl is not methyl, CH₃(CH₂)₁₆—, CH₃(CH₂)₁₄—, CH₃(CH₂)₁₂—, or CH₃(CH₂)₁₀—,

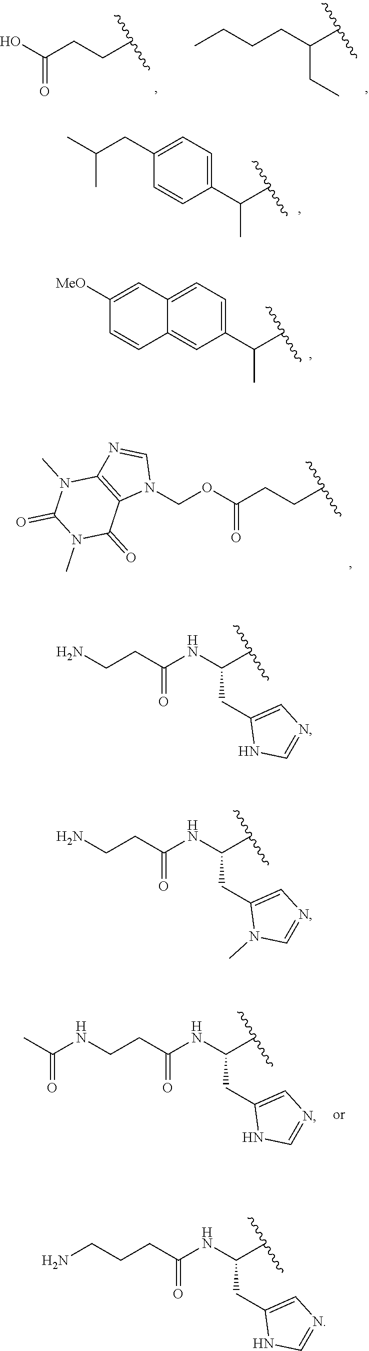

In certain embodiments, useful R¹ alkyl groups include butyl, pentyl, hexyl, heptyl, octyl, nonyl, decyl, and constitutional isomers thereof. In one embodiment, alkyl is butyl, and constitutional isomers thereof. In one embodiment, alkyl is pentyl, and constitutional isomers thereof. In one embodiment, alkyl is hexyl, and constitutional isomers thereof. In one embodiment, alkyl is heptyl, and constitutional isomers thereof, except

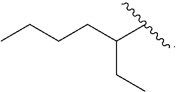 [SMILES: *C(CC)CCCC]

In one embodiment, alkyl is octyl, and constitutional isomers thereof. In one embodiment, alkyl is nonyl, and constitutional isomers thereof. In one embodiment, alkyl is decyl, and constitutional isomers thereof. In one embodiment, R¹ is —(C═O)alkenyl, wherein alkenyl is optionally substituted and/or comprises one or more stereogenic centers. In one embodiment, R¹ is —(C═O)alkynyl, wherein alkynyl is optionally substituted and/or comprises one or more stereogenic centers. In one embodiment, R¹ is —(C═O)cycloalkyl, wherein cycloalkyl is optionally substituted and/or comprises one or more stereogenic centers. In one embodiment, R¹ is —(C═O)alkoxyalkyl, wherein alkoxyalkyl is optionally substituted and/or comprises one or more stereogenic centers. In one embodiment, R¹ is —(C═O)heterocyclyl, wherein heterocyclyl is optionally substituted and/or comprises one or more stereogenic centers, and wherein heterocyclyl is not

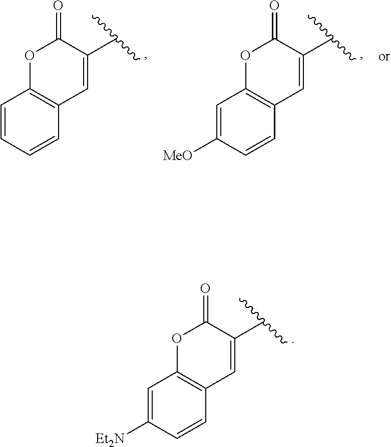

In one embodiment, R¹ is —(C═O)heterocycloalkyl, wherein heterocycloalkyl is optionally substituted and/or comprises one or more stereogenic centers, and wherein heterocycloalkyl is not

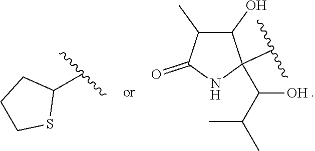 [SMILES: *C1CCCS1 ; *C1(C(O)C(C)C)NC(=O)C(C)C1O]

In one embodiment, R¹ is —(C═O)aryl, wherein aryl is optionally substituted and/or comprises one or more stereogenic centers, and wherein aryl is not phenyl, 2-acetoxyphenyl, 4-nitrophenyl, or 3,4,5-trimethoxyphenyl. In certain embodiments, useful R¹ aryl groups include optionally substituted naphthyl, fluorenyl, azulenyl, anthryl, phenanthryl, and pyrenyl. In one embodiment, aryl is optionally substituted naphthyl. In one embodiment, aryl is optionally substituted fluorenyl. In one embodiment, aryl is optionally substituted azulenyl. In one embodiment, aryl is optionally substituted anthryl. In one embodiment, aryl is optionally substituted phenanthryl. In one embodiment, aryl is optionally substituted pyrenyl. In one embodiment, R¹ is —(C═O)arylalkyl, wherein arylalkyl is optionally substituted and/or comprises one or more stereogenic centers. In one embodiment, R¹ is —(C═O)heteroaryl, wherein heteroaryl is optionally substituted and/or comprises one or more stereogenic centers, and wherein heteroaryl is not 3-pyridyl. In one embodiment, when R¹ is

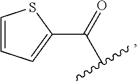 [SMILES: *C(=O)c1cccs1]

then the compound has an ee greater than zero. In any of the embodiments within this paragraph, the compound is a pharmaceutically acceptable salt thereof.

In one embodiment, the compound is

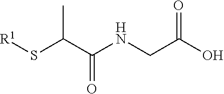

wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl; wherein alkyl, alkenyl, alkynyl, cycloalkyl, aryl, and heteroaryl are optionally substituted and/or comprise one or more nonracemic stereogenic centers; wherein R¹ is not

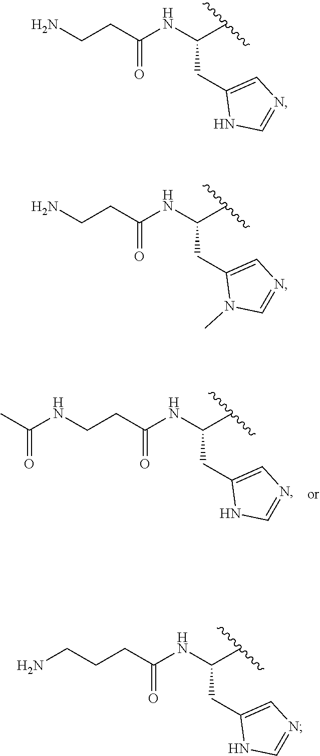 [SMILES: *[C@H](Cc1cnc[nH]1)NC(=O)CCN ; *[C@H](Cc1cncn1C)NC(=O)CCN ; *[C@H](Cc1cnc[nH]1)NC(=O)CCNC(C)=O ; *[C@H](Cc1cnc[nH]1)NC(=O)CCCN]

or a stereoisomer thereof or a pharmaceutically acceptable salt thereof. In one embodiment, R¹ is —(C═O)alkyl, wherein alkyl is optionally substituted and/or comprises one or more nonracemic stereogenic centers. In one embodiment, R¹ is —(C═O)alkyl, wherein alkyl is optionally substituted and/or comprises one or more nonracemic stereogenic centers, and wherein alkyl is not

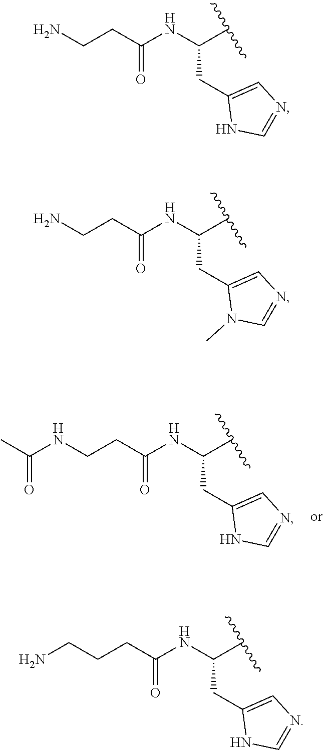 [SMILES: *[C@H](Cc1cnc[nH]1)NC(=O)CCN ; *[C@H](Cc1cncn1C)NC(=O)CCN ; *[C@H](Cc1cnc[nH]1)NC(=O)CCNC(C)=O ; *[C@H](Cc1cnc[nH]1)NC(=O)CCCN]

In another embodiment, R¹ is —(C═O)alkenyl, wherein alkenyl is optionally substituted and/or comprises one or more nonracemic stereogenic centers. In another embodiment, R¹ is —(C═O)alkynyl, wherein alkynyl is optionally substituted and/or comprises one or more nonracemic stereogenic centers. In another embodiment, R¹ is —(C═O)cycloalkyl, wherein cycloalkyl is optionally substituted and/or comprises one or more nonracemic stereogenic centers. In another embodiment, R¹ is —(C═O)alkoxyalkyl, wherein alkoxyalkyl is optionally substituted and/or comprises one or more nonracemic stereogenic centers. In another embodiment, R¹ is —(C═O)heterocyclyl, wherein heterocyclyl is optionally substituted and/or comprises one or more nonracemic stereogenic centers. In another embodiment, R¹ is —(C═O)heterocycloalkyl, wherein heterocycloalkyl is optionally substituted and/or comprises one or more nonracemic stereogenic centers. In another embodiment, R¹ is —(C═O)aryl, wherein aryl is optionally substituted and/or comprises one or more nonracemic stereogenic centers. In another embodiment, R¹ is —(C═O)arylalkyl, wherein arylalkyl is optionally substituted and/or comprises one or more nonracemic stereogenic centers. In another embodiment, R¹ is —(C═O)heteroaryl, wherein heteroaryl is optionally substituted and/or comprises one or more nonracemic stereogenic centers. In any of the embodiments within this paragraph, the compound is a pharmaceutically acceptable salt thereof.

In one embodiment, the compound is

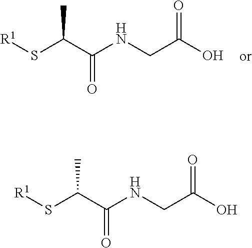

wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl; wherein alkyl, alkenyl, alkynyl, cycloalkyl, aryl, and heteroaryl are optionally substituted and/or comprise one or more stereogenic centers; or a pharmaceutically acceptable salt thereof. In one embodiment, R¹ is —(C═O)alkyl, wherein alkyl is optionally substituted and/or comprises one or more stereogenic centers. In one embodiment, R¹ is —(C═O)alkenyl, wherein alkenyl is optionally substituted and/or comprises one or more stereogenic centers. In one embodiment, R¹ is —(C═O)alkynyl, wherein alkynyl is optionally substituted and/or comprises one or more stereogenic centers. In one embodiment, R¹ is —(C═O)cycloalkyl, wherein cycloalkyl is optionally substituted and/or comprises one or more stereogenic centers. In one embodiment, R¹ is —(C═O)alkoxyalkyl, wherein alkoxyalkyl is optionally substituted and/or comprises one or more stereogenic centers. In one embodiment, R¹ is —(C═O)heterocyclyl, wherein heterocyclyl is optionally substituted and/or comprises one or more stereogenic centers. In one embodiment, R¹ is —(C═O)heterocycloalkyl, wherein heterocycloalkyl is optionally substituted and/or comprises one or more stereogenic centers. In one embodiment, R¹ is —(C═O)aryl, wherein aryl is optionally substituted and/or comprises one or more stereogenic centers. In one embodiment, R¹ is —(C═O)arylalkyl, wherein arylalkyl is optionally substituted and/or comprises one or more stereogenic centers. In one embodiment, R¹ is —(C═O)heteroaryl, wherein heteroaryl is optionally substituted and/or comprises one or more stereogenic centers. In any of the embodiments in this paragraph, the compound is a pharmaceutically acceptable salt thereof.

In one embodiment, the compound is

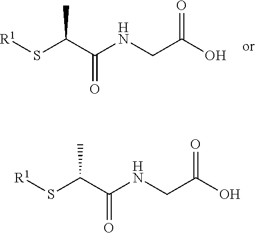

wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl; wherein alkyl, alkenyl, alkynyl, cycloalkyl, aryl, and heteroaryl are optionally substituted and/or comprise one or more nonracemic stereogenic centers; or a pharmaceutically acceptable salt thereof. In one embodiment, R¹ is —(C═O)alkyl, wherein alkyl is optionally substituted and/or comprises one or more nonracemic stereogenic centers. In one embodiment, R¹ is —(C═O)alkenyl, wherein alkenyl is optionally substituted and/or comprises one or more nonracemic stereogenic centers. In one embodiment, R¹ is —(C═O)alkynyl, wherein alkynyl is optionally substituted and/or comprises one or more nonracemic stereogenic centers. In one embodiment, R¹ is —(C═O)cycloalkyl, wherein cycloalkyl is optionally substituted and/or comprises one or more nonracemic stereogenic centers. In one embodiment, R¹ is —(C═O)alkoxyalkyl, wherein alkoxyalkyl is optionally substituted and/or comprises one or more nonracemic stereogenic centers. In one embodiment, R¹ is —(C═O)heterocyclyl, wherein heterocyclyl is optionally substituted and/or comprises one or more nonracemic stereogenic centers. In one embodiment, R¹ is —(C═O)heterocycloalkyl, wherein heterocycloalkyl is optionally substituted and/or comprises one or more nonracemic stereogenic centers. In one embodiment, R¹ is —(C═O)aryl, wherein aryl is optionally substituted and/or comprises one or more nonracemic stereogenic centers. In one embodiment, R¹ is —(C═O)arylalkyl, wherein arylalkyl is optionally substituted and/or comprises one or more nonracemic stereogenic centers. In one embodiment, R¹ is —(C═O)heteroaryl, wherein heteroaryl is optionally substituted and/or comprises one or more nonracemic stereogenic centers. In any of the embodiments in this paragraph, the compound is a pharmaceutically acceptable salt thereof.

In certain embodiments, the compound is

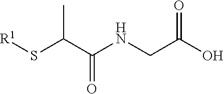

wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl; wherein alkyl, alkenyl, alkynyl, cycloalkyl, aryl, and heteroaryl are optionally substituted and/or comprise one or more stereogenic centers; wherein the compound has an ee or de greater than zero; or a pharmaceutically acceptable salt thereof. In one embodiment, R¹ is —(C═O)alkyl, wherein alkyl is optionally substituted and/or comprises one or more stereogenic centers, and wherein the compound has an ee or de greater than zero. In one embodiment, R¹ is —(C═O)alkenyl, wherein alkenyl is optionally substituted and/or comprises one or more stereogenic centers, and wherein the compound has an ee or de greater than zero. In one embodiment, R¹ is —(C═O)alkynyl, wherein alkynyl is optionally substituted and/or comprises one or more stereogenic centers, and wherein the compound has an ee or de greater than zero. In one embodiment, R¹ is —(C═O)cycloalkyl, wherein cycloalkyl is optionally substituted and/or comprises one or more stereogenic centers, and wherein the compound has an ee or de greater than zero. In one embodiment, R¹ is —(C═O)alkoxyalkyl, wherein alkoxyalkyl is optionally substituted and/or comprises one or more stereogenic centers, and wherein the compound has an ee or de greater than zero. In one embodiment, R¹ is —(C═O)heterocyclyl, wherein heterocyclyl is optionally substituted and/or comprises one or more stereogenic centers, and wherein the compound has an ee or de greater than zero. In one embodiment, R¹ is —(C═O)heterocycloalkyl, wherein heterocycloalkyl is optionally substituted and/or comprises one or more stereogenic centers, and wherein the compound has an ee or de greater than zero. In one embodiment, R¹ is —(C═O)aryl, wherein aryl is optionally substituted and/or comprises one or more stereogenic centers, and wherein the compound has an ee or de greater than zero. In one embodiment, R¹ is —(C═O)arylalkyl, wherein arylalkyl is optionally substituted and/or comprises one or more stereogenic centers, and wherein the compound has an ee or de greater than zero. In one embodiment, R¹ is —(C═O)heteroaryl, wherein heteroaryl is optionally substituted and/or comprises one or more stereogenic centers, and wherein the compound has an ee or de greater than zero. In any of the embodiments in this paragraph, the compound is a pharmaceutically acceptable salt thereof.

In certain embodiments, when alkyl, alkenyl, alkynyl, cycloalkyl, aryl, or heteroaryl is substituted, then the substituents are selected from the group consisting of alkyl, alkenyl, alkynyl, cycloalkyl, aryl, heteroaryl, amino, halo, cyano, nitro, hydroxy, acyl, amido, —C(O)—, —C(S)—, haloalkyl, and azido.

In certain embodiments, the compound is

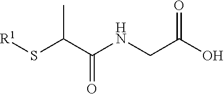

wherein when R¹ is —(C═O)alkyl, then alkyl is methyl, CH₃(CH₂)₁₆—, CH₃(CH₂)₁₄—, CH₃(CH₂)₁₂—, CH₃(CH₂)₁₀—

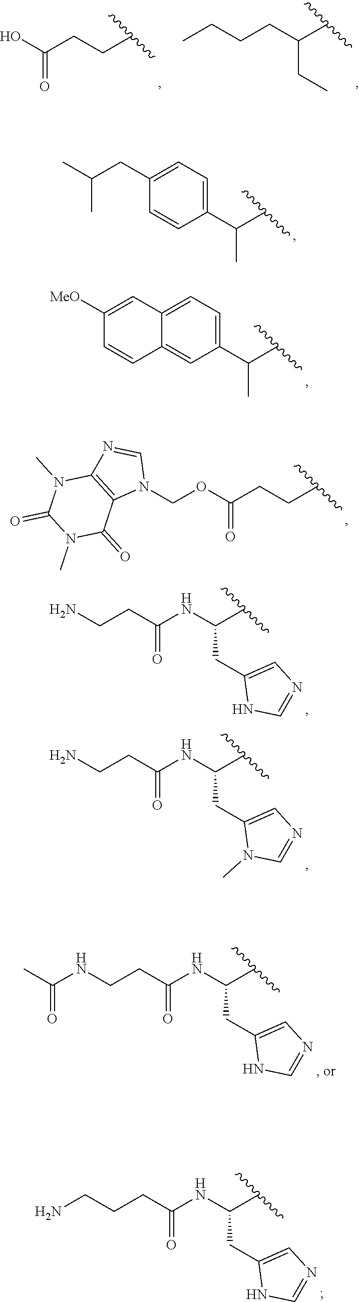

or wherein when R¹ is —(C═O)heterocycloalkyl, then heterocycloalkyl is

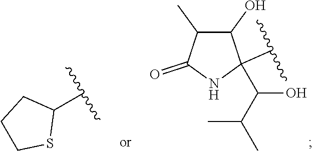 [SMILES: *C1CCCS1 ; *C1(C(O)C(C)C)NC(=O)C(C)C1O]

and wherein the compound has an ee or de greater than zero. In one embodiment, alkyl is methyl and the compound has an ee greater than zero. In one embodiment, alkyl is CH₃(CH₂)₁₆— and the compound has an ee greater than zero. In one embodiment, alkyl is CH₃(CH₂)₁₄— and the compound has an ee greater than zero. In one embodiment, alkyl is CH₃(CH₂)₁₂— and the compound has an ee greater than zero. In one embodiment, alkyl is CH₃(CH₂)₁₀— and the compound has an ee greater than zero. In one embodiment, alkyl is

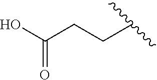 [SMILES: *CCC(=O)O]

and the compound has an ee greater than zero. In one embodiment, alkyl is

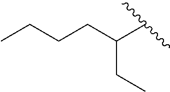 [SMILES: *C(CC)CCCC]

and the compound has an ee or de greater than zero. In one embodiment, alkyl is

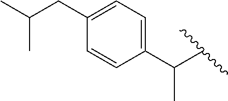 [SMILES: *C(C)c1ccc(CC(C)C)cc1]

and the compound has an ee or de greater than zero. In one embodiment, alkyl is

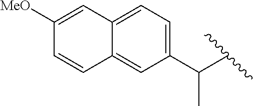 [SMILES: *C(C)c1ccc2cc(OC)ccc2c1]

and the compound has an ee or de greater than zero. In one embodiment, alkyl is

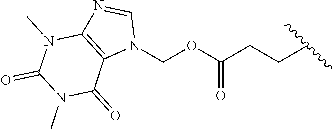 [SMILES: *CCC(=O)OCn1cnc2c1c(=O)n(C)c(=O)n2C]

and the compound has an ee greater than zero. In one embodiment, alkyl is

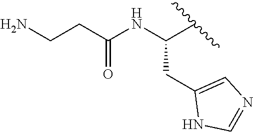 [SMILES: *[C@H](Cc1cnc[nH]1)NC(=O)CCN]

and the compound has an ee or de greater than zero. In one embodiment, alkyl is

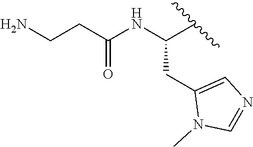 [SMILES: *[C@H](Cc1cncn1C)NC(=O)CCN]

and the compound has an ee or de greater than zero. In one embodiment, alkyl is

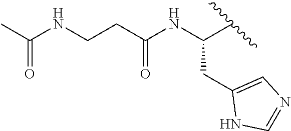 [SMILES: *[C@H](Cc1cnc[nH]1)NC(=O)CCNC(C)=O]

and the compound has an ee or de greater than zero. In one embodiment, alkyl is

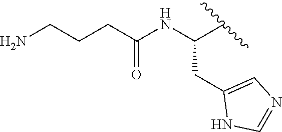 [SMILES: *[C@H](Cc1cnc[nH]1)NC(=O)CCCN]

and the compound has an ee or de greater than zero. In one embodiment, heterocycloalkyl is

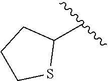 [SMILES: *C1CCCS1]

and the compound has an ee or de greater than zero. In one embodiment, heterocycloalkyl is

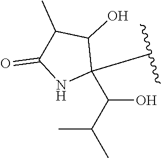 [SMILES: *C1(C(O)C(C)C)NC(=O)C(C)C1O]

and the compound has an ee or de greater than zero. In any of the embodiments in this paragraph, the compound is a pharmaceutically acceptable salt thereof.

In one embodiment, the compound is

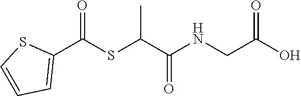 [SMILES: CC(SC(=O)c1cccs1)C(=O)NCC(=O)O]

having an ee or % ee greater than zero. In another embodiment, the compound is

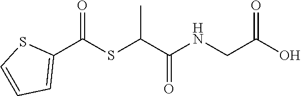 [SMILES: CC(SC(=O)c1cccs1)C(=O)NCC(=O)O]

where the ee or % ee is a range from ninety to one hundred. In another embodiment, the compound is

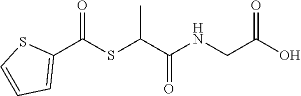 [SMILES: CC(SC(=O)c1cccs1)C(=O)NCC(=O)O]

where the ee or % ee is a range from ninety-five to one hundred. In another embodiment, the compound is

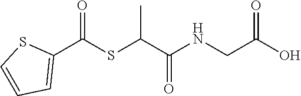 [SMILES: CC(SC(=O)c1cccs1)C(=O)NCC(=O)O]

where the ee or % ee is a range from ninety-seven to one hundred. In another embodiment, the compound is

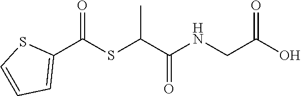 [SMILES: CC(SC(=O)c1cccs1)C(=O)NCC(=O)O]

where the ee or % ee is a range from ninety-eight to one hundred. In another embodiment, the compound is

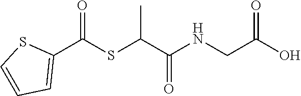 [SMILES: CC(SC(=O)c1cccs1)C(=O)NCC(=O)O]

where the ee or % ee is a range from ninety-nine to one hundred. In any of the embodiments within this paragraph, the compound is a pharmaceutically acceptable salt thereof.

In one embodiment, the compound is

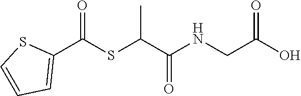 [SMILES: CC(SC(=O)c1cccs1)C(=O)NCC(=O)O]

having an ee greater than zero. In another embodiment, the compound is

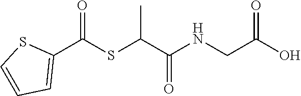 [SMILES: CC(SC(=O)c1cccs1)C(=O)NCC(=O)O]

where the ee or % ee is an integer from one to one hundred. In one embodiment, the ee or % ee is one. In one embodiment, the ee or % ee is two. In one embodiment, the ee or % ee is three. In one embodiment, the ee or % ee is four. In one embodiment, the ee or % ee is five. In one embodiment, the ee or % ee is six. In one embodiment, the ee or % ee is seven. In one embodiment, the ee or % ee is eight. In one embodiment, the ee or % ee is nine. In one embodiment, the ee or % ee is ten. In one embodiment, the ee or % ee is eleven. In one embodiment, the ee or % ee is twelve. In one embodiment, the ee or % ee is thirteen. In one embodiment, the ee or % ee is fourteen. In one embodiment, the ee or % ee is fifteen. In one embodiment, the ee or % ee is sixteen. In one embodiment, the ee or % ee is seventeen. In one embodiment, the ee or % ee is eighteen. In one embodiment, the ee or % ee is nineteen. In one embodiment, the ee or % ee is twenty. In one embodiment, the ee or % ee is twenty-one. In one embodiment, the ee or % ee is twenty-two. In one embodiment, the ee or % ee is twenty-three. In one embodiment, the ee or % ee is twenty-four. In one embodiment, the ee or % ee is twenty-five. In one embodiment, the ee or % ee is twenty-six. In one embodiment, the ee or % ee is twenty-seven. In one embodiment, the ee or % ee is twenty-eight. In one embodiment, the ee or % ee is twenty-nine. In one embodiment, the ee or % ee is thirty. In one embodiment, the ee or % ee is thirty-one. In one embodiment, the ee or % ee is thirty-two. In one embodiment, the ee or % ee is thirty-three. In one embodiment, the ee or % ee is thirty-four. In one embodiment, the ee or % ee is thirty-five. In one embodiment, the ee or % ee is thirty-six. In one embodiment, the ee or % ee is thirty-seven. In one embodiment, the ee or % ee is thirty-eight. In one embodiment, the ee or % ee is thirty-nine. In one embodiment, the ee or % ee is forty. In one embodiment, the ee or % ee is forty-one. In one embodiment, the ee or % ee is forty-two. In one embodiment, the ee or % ee is forty-three. In one embodiment, the ee or % ee is forty-four. In one embodiment, the ee or % ee is forty-five. In one embodiment, the ee or % ee is forty-six. In one embodiment, the ee or % ee is forty-seven. In one embodiment, the ee or % ee is forty-eight. In one embodiment, the ee or % ee is forty-nine. In one embodiment, the ee or % ee is fifty. In one embodiment, the ee or % ee is fifty-one. In one embodiment, the ee or % ee is fifty-two. In one embodiment, the ee or % ee is fifty-three. In one embodiment, the ee or % ee is fifty-four. In one embodiment, the ee or % ee is fifty-five. In one embodiment, the ee or % ee is fifty-six. In one embodiment, the ee or % ee is fifty-seven. In one embodiment, the ee or % ee is fifty-eight. In one embodiment, the ee or % ee is fifty-nine. In one embodiment, the ee or % ee is sixty. In one embodiment, the ee or % ee is sixty-one. In one embodiment, the ee or % ee is sixty-two. In one embodiment, the ee or % ee is sixty-three. In one embodiment, the ee or % ee is sixty-four. In one embodiment, the ee or % ee is sixty-five. In one embodiment, the ee or % ee is sixty-six. In one embodiment, the ee or % ee is sixty-seven. In one embodiment, the ee or % ee is sixty-eight. In one embodiment, the ee or % ee is sixty-nine. In one embodiment, the ee or % ee is seventy. In one embodiment, the ee or % ee is seventy-one. In one embodiment, the ee or % ee is seventy-two. In one embodiment, the ee or % ee is seventy-three. In one embodiment, the ee or % ee is seventy-four. In one embodiment, the ee or % ee is seventy-five. In one embodiment, the ee or % ee is seventy-six. In one embodiment, the ee or % ee is seventy-seven. In one embodiment, the ee or % ee is seventy-eight. In one embodiment, the ee or % ee is seventy-nine. In one embodiment, the ee or % ee is eighty. In one embodiment, the ee or % ee is eighty-one. In one embodiment, the ee or % ee is eighty-two. In one embodiment, the ee or % ee is eighty-three. In one embodiment, the ee or % ee is eighty-four. In one embodiment, the ee or % ee is eighty-five. In one embodiment, the ee or % ee is eighty-six. In one embodiment, the ee or % ee is eighty-seven. In one embodiment, the ee or % ee is eighty-eight. In one embodiment, the ee or % ee is eighty-nine. In one embodiment, the ee or % ee is ninety. In one embodiment, the ee or % ee is ninety-one. In one embodiment, the ee or % ee is ninety-two. In one embodiment, the ee or % ee is ninety-three. In one embodiment, the ee or % ee is ninety-four. In one embodiment, the ee or % ee is ninety-five. In one embodiment, the ee or % ee is ninety-six. In one embodiment, the ee or % ee is ninety-seven. In one embodiment, the ee or % ee is ninety-eight. In one embodiment, the ee or % ee is ninety-nine. In one embodiment, the ee or % ee is one hundred. In one embodiment, the compound is

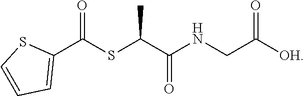 [SMILES: C[C@H](SC(=O)c1cccs1)C(=O)NCC(=O)O]

In one embodiment, the compound is

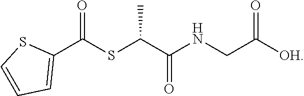 [SMILES: C[C@@H](SC(=O)c1cccs1)C(=O)NCC(=O)O]

In any of the embodiments within this paragraph, the compound is a pharmaceutically acceptable salt thereof.

In certain embodiments, the compound is

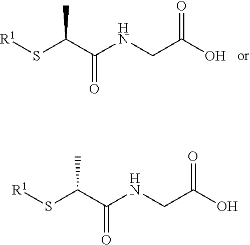

wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl; wherein alkyl, alkenyl, alkynyl, cycloalkyl, aryl, and heteroaryl are optionally substituted and/or comprise one or more nonracemic stereogenic centers. In certain embodiments, these compounds have an ee, de, % ee, or % de range from ninety to one hundred. In one embodiment, the ee, de, % ee, or % de range is from ninety-five to one hundred. In one embodiment, the ee, de, % ee, or % de range is from ninety-seven to one hundred. In one embodiment, the ee, de, % ee, or % de range is from ninety-eight to one hundred. In one embodiment, the ee, de, % ee, or % de range is from ninety-nine to one hundred. In any of the embodiments in this paragraph, the compound is a pharmaceutically acceptable salt thereof.

In certain embodiments, the compound is

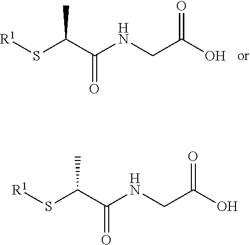

wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl; wherein alkyl, alkenyl, alkynyl, cycloalkyl, aryl, and heteroaryl are optionally substituted and/or comprise one or more nonracemic stereogenic centers. In certain embodiments, these compounds have an ee, de, % ee, or % de that is an integer from one to one hundred. In one embodiment, the ee, de, % ee, or % de is one. In one embodiment, the ee, de, % ee, or % de is two. In one embodiment, the ee, de, % ee, or % de is three. In one embodiment, the ee, de, % ee, or % de is four. In one embodiment, the ee, de, % ee, or % de is five. In one embodiment, the ee, de, % ee, or % de is six. In one embodiment, the ee, de, % ee, or % de is seven. In one embodiment, the ee, de, % ee, or % de is eight. In one embodiment, the ee, de, % ee, or % de is nine. In one embodiment, the ee, de, % ee, or % de is ten. In one embodiment, the ee, de, % ee, or % de is eleven. In one embodiment, the ee, de, % ee, or % de is twelve. In one embodiment, the ee, de, % ee, or % de is thirteen. In one embodiment, the ee, de, % ee, or % de is fourteen. In one embodiment, the ee, de, % ee, or % de is fifteen. In one embodiment, the ee, de, % ee, or % de is sixteen. In one embodiment, the ee, de, % ee, or % de is seventeen. In one embodiment, the ee, de, % ee, or % de is eighteen. In one embodiment, the ee, de, % ee, or % de is nineteen. In one embodiment, the ee, de, % ee, or % de is twenty. In one embodiment, the ee, de, % ee, or % de is twenty-one. In one embodiment, the ee, de, % ee, or % de is twenty-two. In one embodiment, the ee, de, % ee, or % de is twenty-three. In one embodiment, the ee, de, % ee, or % de is twenty-four. In one embodiment, the ee, de, % ee, or % de is twenty-five. In one embodiment, the ee, de, % ee, or % de is twenty-six. In one embodiment, the ee, de, % ee, or % de is twenty-seven. In one embodiment, the ee, de, % ee, or % de is twenty-eight. In one embodiment, the ee, de, % ee, or % de is twenty-nine. In one embodiment, the ee, de, % ee, or % de is thirty. In one embodiment, the ee, de, % ee, or % de is thirty-one. In one embodiment, the ee, de, % ee, or % de is thirty-two. In one embodiment, the ee, de, % ee, or % de is thirty-three. In one embodiment, the ee, de, % ee, or % de is thirty-four. In one embodiment, the ee, de, % ee, or % de is thirty-five. In one embodiment, the ee, de, % ee, or % de is thirty-six. In one embodiment, the ee, de, % ee, or % de is thirty-seven. In one embodiment, the ee, de, % ee, or % de is thirty-eight. In one embodiment, the ee, de, % ee, or % de is thirty-nine. In one embodiment, the ee, de, % ee, or % de is forty. In one embodiment, the ee, de, % ee, or % de is forty-one. In one embodiment, the ee, de, % ee, or % de is forty-two. In one embodiment, the ee, de, % ee, or % de is forty-three. In one embodiment, the ee, de, % ee, or % de is forty-four. In one embodiment, the ee, de, % ee, or % de is forty-five. In one embodiment, the ee, de, % ee, or % de is forty-six. In one embodiment, the ee, de, % ee, or % de is forty-seven. In one embodiment, the ee, de, % ee, or % de is forty-eight. In one embodiment, the ee, de, % ee, or % de is forty-nine. In one embodiment, the ee, de, % ee, or % de is fifty. In one embodiment, the ee, de, % ee, or % de is fifty-one. In one embodiment, the ee, de, % ee, or % de is fifty-two. In one embodiment, the ee, de, % ee, or % de is fifty-three. In one embodiment, the ee, de, % ee, or % de is fifty-four. In one embodiment, the ee, de, % ee, or % de is fifty-five. In one embodiment, the ee, de, % ee, or % de is fifty-six. In one embodiment, the ee, de, % ee, or % de is fifty-seven. In one embodiment, the ee, de, % ee, or % de is fifty-eight. In one embodiment, the ee, de, % ee, or % de is fifty-nine. In one embodiment, the ee, de, % ee, or % de is sixty. In one embodiment, the ee, de, % ee, or % de is sixty-one. In one embodiment, the ee, de, % ee, or % de is sixty-two. In one embodiment, the ee, de, % ee, or % de is sixty-three. In one embodiment, the ee, de, % ee, or % de is sixty-four. In one embodiment, the ee, de, % ee, or % de is sixty-five. In one embodiment, the ee, de, % ee, or % de is sixty-six. In one embodiment, the ee, de, % ee, or % de is sixty-seven. In one embodiment, the ee, de, % ee, or % de is sixty-eight. In one embodiment, the ee, de, % ee, or % de is sixty-nine. In one embodiment, the ee, de, % ee, or % de is seventy. In one embodiment, the ee, de, % ee, or % de is seventy-one. In one embodiment, the ee, de, % ee, or % de is seventy-two. In one embodiment, the ee, de, % ee, or % de is seventy-three. In one embodiment, the ee, de, % ee, or % de is seventy-four. In one embodiment, the ee, de, % ee, or % de is seventy-five. In one embodiment, the ee, de, % ee, or % de is seventy-six. In one embodiment, the ee, de, % ee, or % de is seventy-seven. In one embodiment, the ee, de, % ee, or % de is seventy-eight. In one embodiment, the ee, de, % ee, or % de is seventy-nine. In one embodiment, the ee, de, % ee, or % de is eighty. In one embodiment, the ee, de, % ee, or % de is eighty-one. In one embodiment, the ee, de, % ee, or % de is eighty-two. In one embodiment, the ee, de, % ee, or % de is eighty-three. In one embodiment, the ee, de, % ee, or % de is eighty-four. In one embodiment, the ee, de, % ee, or % de is eighty-five. In one embodiment, the ee, de, % ee, or % de is eighty-six. In one embodiment, the ee, de, % ee, or % de is eighty-seven. In one embodiment, the ee, de, % ee, or % de is eighty-eight. In one embodiment, the ee, de, % ee, or % de is eighty-nine. In one embodiment, the ee, de, % ee, or % de is ninety. In one embodiment, the ee, de, % ee, or % de is ninety-one. In one embodiment, the ee, de, % ee, or % de is ninety-two. In one embodiment, the ee, de, % ee, or % de is ninety-three. In one embodiment, the ee, de, % ee, or % de is ninety-four. In one embodiment, the ee, de, % ee, or % de is ninety-five. In one embodiment, the ee, de, % ee, or % de is ninety-six. In one embodiment, the ee, de, % ee, or % de is ninety-seven. In one embodiment, the ee, de, % ee, or % de is ninety-eight. In one embodiment, the ee, de, % ee, or % de is ninety-nine. In one embodiment, the ee, de, % ee, or % de is one hundred. In any of the embodiments in this paragraph, the compound is a pharmaceutically acceptable salt thereof.

### Methods of Preparing Compounds or Thioesters

The compounds provided herein can be prepared, isolated, or obtained by any method apparent to those of skill in the art. In certain embodiments, compounds provided herein can generally be prepared according to Scheme A.

### Scheme A. Exemplary Preparation Scheme

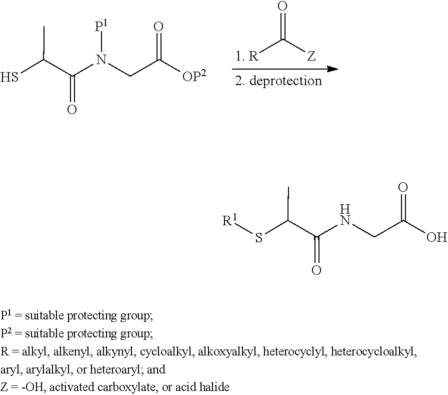

In Scheme A, R¹ is described in the context of compounds described or contemplated herein. Mercaptans can be acylated. The protected thioesters can then be deprotected under standard conditions, as would be appreciated by a person of skill in the art. Suitable reaction conditions are well known to those in the art.

In certain embodiments, compounds provided herein that include at least one chiral center can generally be separated into single enantiomeric or single diastereomeric forms according to general chiral separation methods available in the art. For example in one embodiment,

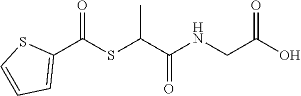 [SMILES: CC(SC(=O)c1cccs1)C(=O)NCC(=O)O]

can be separated into

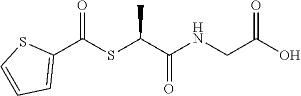 [SMILES: C[C@H](SC(=O)c1cccs1)C(=O)NCC(=O)O]

via crystallization, chemical or enzymatic kinetic resolution, chiral reagents or chiral auxillaries, chromatography, or chiral chromatography. For example, in one embodiment,

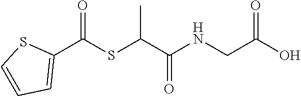 [SMILES: CC(SC(=O)c1cccs1)C(=O)NCC(=O)O]

can be separated into

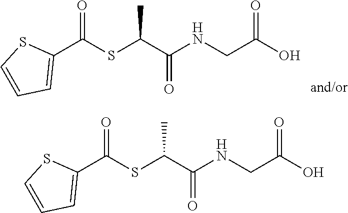

via crystallization techniques. By way of further example, in one embodiment,

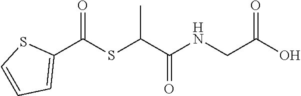 [SMILES: CC(SC(=O)c1cccs1)C(=O)NCC(=O)O]

can be separated into

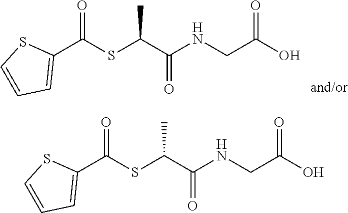

via chemical kinetic resolution. By way of further example, in one embodiment,

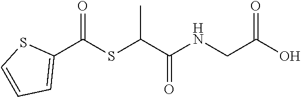 [SMILES: CC(SC(=O)c1cccs1)C(=O)NCC(=O)O]

can be separated into

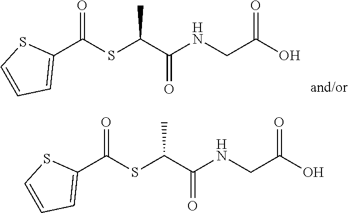

via enzymatic kinetic resolution. By way of further example, in one embodiment,

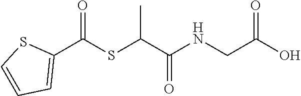 [SMILES: CC(SC(=O)c1cccs1)C(=O)NCC(=O)O]

can be separated into

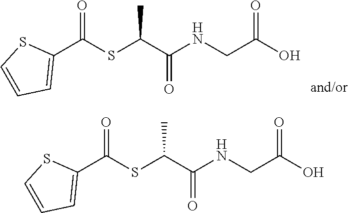

via diastereomer formation via reaction with a chiral reagent or chiral auxillary from the chiral pool. Exemplary chiral reagents or chiral auxillaries, stoichiometric or catalytic, for such transformations include, without limitation, nonracemic menthol esterifications and transacylation processes with chiral catalysts. By way of further example, chromatography can be via standard flash chromatography following suitable diastereomer formation. By way of further example, chromatography can be via HPLC or GCMS methods following suitable diastereomer formation using suitable stationary phases purchased from suitable vendors. By way of further example, chiral chromatography can be via chiral HPLC or GCMS methods using suitable chiral stationary phases purchased from suitable vendors. In any embodiment in this paragraph that utilizes diastereomeric forms, a person of skill in the art should appreciate that the separated diastereomer can be reconverted into the enantiomeric carboxlic acid form following the separation methods described herein.

### Pharmaceutical Compositions and Methods of Treatment

Provided herein are methods of treating and preventing diseases, conditions, or disorders comprising administering a therapeutically or prophylactically effective amount of one or more of the compounds disclosed herein, for example, one or more of the compounds of a formula provided herein. Diseases, disorders, and/or conditions include, but are not limited to, renal anomalies. Routes of administration include, without limitation, oral, intravenous, nasal, and/or parenteral injection or infusion modes.

The compounds described herein can be administered alone or together with one or more additional therapeutic agents. The one or more additional therapeutic agents can be administered just prior to, concurrent with, or shortly after the administration of the compounds described herein. The present disclosure also includes pharmaceutical compositions comprising any of the compounds described or contemplated herein, or pharmaceutically acceptable salts thereof. The present disclosure also includes pharmaceutical compositions comprising any of the compounds described or contemplated herein in combination with one or more additional therapeutic agents, and methods of treatment comprising administering such combinations to subjects in need thereof.

Suitable additional therapeutic agents include, but are not limited to, a second thioester akin to those described herein or otherwise, a hormone, a biologic, or a monoclonal antibody. Suitable therapeutic agents also include, but are not limited to, any pharmaceutically acceptable salts, acids, or derivatives of a compound set forth herein.

In some embodiments of the methods described herein, multiple doses of a compound described herein (or a pharmaceutical composition comprising a combination of a compound described herein and any of the additional therapeutic agents contemplated herein) may be administered to a subject over a defined time course. The methods according to this embodiment of the disclosure comprise sequentially administering to a subject multiple doses of a compound described herein. As used herein, “sequentially administering” means that each dose of the compound is administered to the subject at a different point in time, e.g., on different days separated by a predetermined interval (e.g., hours, days, weeks, or months). The present disclosure includes methods which comprise sequentially administering to the patient a single initial dose of a compound described herein, followed by one or more secondary doses of the compound, and optionally followed by one or more tertiary doses of the compound.

The terms “initial dose,” “secondary doses,” and “tertiary doses,” refer to the temporal sequence of administration of the compounds described herein. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses can all include the same amount of the compound described herein, but generally can differ from one another in terms of frequency of administration. In certain embodiments, the amount of the compound included in the initial, secondary, and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present disclosure, each secondary and/or tertiary dose is administered one to twenty-six (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of the compound which is administered to a patient prior to the administration of the very next dose in the sequence with no intervening doses.

The methods according to this embodiment of the disclosure may comprise administering to a patient any number of secondary and/or tertiary doses of the compound. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient. The administration regimen may be carried out indefinitely over the lifetime of a particular subject, or until such treatment is no longer therapeutically needed or advantageous.

In embodiments involving multiple secondary doses, each secondary dose may be administered at the same frequency as the other secondary doses. For example, each secondary dose may be administered to the patient one to two weeks or one to two months after the immediately preceding dose. Similarly, in embodiments involving multiple tertiary doses, each tertiary dose may be administered at the same frequency as the other tertiary doses. For example, each tertiary dose may be administered to the patient two to twelve weeks after the immediately preceding dose. In certain embodiments of the disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

The present disclosure includes administration regimens in which two to six loading doses are administered to a patient at a first frequency (e.g., once a week, once every two weeks, once every three weeks, once a month, once every two months, etc.), followed by administration of two or more maintenance doses to the patient on a less frequent basis. For example, according to this embodiment of the disclosure, if the loading doses are administered at a frequency of once a month, then the maintenance doses may be administered to the patient once every six weeks, once every two months, once every three months, etc.

The present disclosure includes pharmaceutical compositions of the compounds or thioesters described and/or contemplated herein, e.g., compositions comprising a compound described or contemplated herein, a salt, stereoisomer, or polymorph thereof, and a pharmaceutically acceptable carrier, diluent, and/or excipient. Examples of suitable carriers, diluents and excipients include, but are not limited to, buffers for maintenance of proper composition pH (e.g., citrate buffers, succinate buffers, acetate buffers, phosphate buffers, lactate buffers, oxalate buffers, and the like), carrier proteins (e.g., human serum albumin), saline, polyols (e.g., trehalose, sucrose, xylitol, sorbitol, and the like), surfactants (e.g., polysorbate 20, polysorbate 80, polyoxolate, and the like), antimicrobials, and antioxidants.

In certain embodiments, the method for treating a condition, disease, or disorder in a patient comprises administering to the patient a therapeutically effective amount of a compound

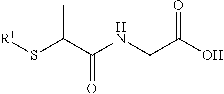

wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl; wherein alkyl, alkenyl, alkynyl, cycloalkyl, aryl, and heteroaryl are optionally substituted and/or comprise one or more nonracemic stereogenic centers. In one embodiment, R¹ is —(C═O)alkyl, wherein alkyl is optionally substituted and/or comprises one or more nonracemic stereogenic centers. In one embodiment, R¹ is —(C═O)alkenyl, wherein alkenyl is optionally substituted and/or comprises one or more nonracemic stereogenic centers. In one embodiment, R¹ is —(C═O)alkynyl, wherein alkynyl is optionally substituted and/or comprises one or more nonracemic stereogenic centers. In one embodiment, R¹ is —(C═O)cycloalkyl, wherein cycloalkyl is optionally substituted and/or comprises one or more nonracemic stereogenic centers. In one embodiment, R¹ is —(C═O)alkoxyalkyl, wherein alkoxyalkyl is optionally substituted and/or comprises one or more nonracemic stereogenic centers. In one embodiment, R¹ is —(C═O)heterocyclyl, wherein heterocyclyl is optionally substituted and/or comprises one or more nonracemic stereogenic centers. In one embodiment, R¹ is —(C═O)heterocycloalkyl, wherein heterocycloalkyl is optionally substituted and/or comprises one or more nonracemic stereogenic centers. In one embodiment, R¹ is —(C═O)aryl, wherein aryl is optionally substituted and/or comprises one or more nonracemic stereogenic centers. In one embodiment, R¹ is —(C═O)arylalkyl, wherein arylalkyl is optionally substituted and/or comprises one or more nonracemic stereogenic centers. In one embodiment, R¹ is —(C═O)heteroaryl, wherein heteroaryl is optionally substituted and/or comprises one or more nonracemic stereogenic centers. In any of the embodiments in this paragraph, the compound is a pharmaceutically acceptable salt thereof.

In certain embodiments, the method for treating a condition, disease, or disorder in a patient comprises administering to the patient a therapeutically effective amount of a compound

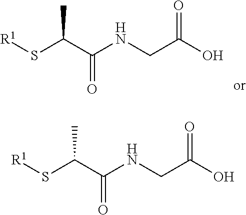

wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl; wherein alkyl, alkenyl, alkynyl, cycloalkyl, aryl, and heteroaryl are optionally substituted and/or comprise one or more stereogenic centers. In one embodiment, R¹ is —(C═O)alkyl, wherein alkyl is optionally substituted and/or comprises one or more stereogenic centers. In one embodiment, R¹ is —(C═O)alkenyl, wherein alkenyl is optionally substituted and/or comprises one or more stereogenic centers. In one embodiment, R¹ is —(C═O)alkynyl, wherein alkynyl is optionally substituted and/or comprises one or more stereogenic centers. In one embodiment, R¹ is —(C═O)cycloalkyl, wherein cycloalkyl is optionally substituted and/or comprises one or more stereogenic centers. In one embodiment, R¹ is —(C═O)alkoxyalkyl, wherein alkoxyalkyl is optionally substituted and/or comprises one or more stereogenic centers. In one embodiment, R¹ is —(C═O)heterocyclyl, wherein heterocyclyl is optionally substituted and/or comprises one or more stereogenic centers. In one embodiment, R¹ is —(C═O)heterocycloalkyl, wherein heterocycloalkyl is optionally substituted and/or comprises one or more stereogenic centers. In one embodiment, R¹ is —(C═O)aryl, wherein aryl is optionally substituted and/or comprises one or more stereogenic centers. In one embodiment, R¹ is —(C═O)arylalkyl, wherein arylalkyl is optionally substituted and/or comprises one or more stereogenic centers. In one embodiment, R¹ is —(C═O)heteroaryl, wherein heteroaryl is optionally substituted and/or comprise one or more stereogenic centers. In any of the embodiments in this paragraph, the compound is a pharmaceutically acceptable salt thereof.

In certain embodiments, the method for treating a condition, disease, or disorder in a patient comprises administering to the patient a therapeutically effective amount of a compound

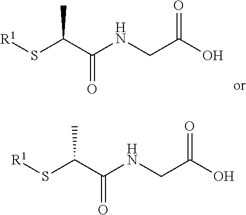

wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl; wherein alkyl, alkenyl, alkynyl, cycloalkyl, aryl, and heteroaryl are optionally substituted and/or comprise one or more nonracemic stereogenic centers. In one embodiment, R¹ is —(C═O)alkyl, wherein alkyl is optionally substituted and/or comprises one or more nonracemic stereogenic centers. In one embodiment, R¹ is —(C═O)alkenyl, wherein alkenyl is optionally substituted and/or comprises one or more nonracemic stereogenic centers. In one embodiment, R¹ is —(C═O)alkynyl, wherein alkynyl is optionally substituted and/or comprises one or more nonracemic stereogenic centers. In one embodiment, R¹ is —(C═O)cycloalkyl, wherein cycloalkyl is optionally substituted and/or comprises one or more nonracemic stereogenic centers. In one embodiment, R¹ is —(C═O)alkoxyalkyl, wherein alkoxyalkyl is optionally substituted and/or comprises one or more nonracemic stereogenic centers. In one embodiment, R¹ is —(C═O)heterocyclyl, wherein heterocyclyl is optionally substituted and/or comprises one or more nonracemic stereogenic centers. In one embodiment, R¹ is —(C═O)heterocycloalkyl, wherein heterocycloalkyl is optionally substituted and/or comprises one or more nonracemic stereogenic centers. In one embodiment, R¹ is —(C═O)aryl, wherein aryl is optionally substituted and/or comprises one or more nonracemic stereogenic centers. In one embodiment, R¹ is —(C═O)arylalkyl, wherein arylalkyl is optionally substituted and/or comprises one or more nonracemic stereogenic centers. In one embodiment, R¹ is —(C═O)heteroaryl, wherein heteroaryl is optionally substituted and/or comprises one or more nonracemic stereogenic centers. In any of the embodiments in this paragraph, the compound is a pharmaceutically acceptable salt thereof. In one embodiment, the compound is

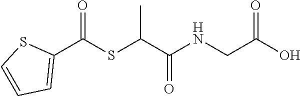 [SMILES: CC(SC(=O)c1cccs1)C(=O)NCC(=O)O]

having an ee greater than zero, as described elsewhere herein. In one embodiment, the compound is

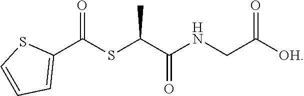 [SMILES: C[C@H](SC(=O)c1cccs1)C(=O)NCC(=O)O]

In one embodiment, the compound is

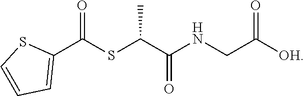 [SMILES: C[C@@H](SC(=O)c1cccs1)C(=O)NCC(=O)O]

In certain embodiments, the condition, disease, or disorder is selected from the group consisting of a kidney disease, a renal disease, a bladder disease, a urinary disease, a ureteral disease, kidney stones, staghorn stones, cystine stones, calculi, lithiasis, nephrolithiasis, urolithiasis, urothiasis, ureterolithiasis, renolithiasis, SLC3A1 gene mutations, SLC7A9 gene mutations, defects in the dibasic amino acid transporter, and cystinuria; wherein when the condition, disease, or disorder is kidney stones, the compound is not

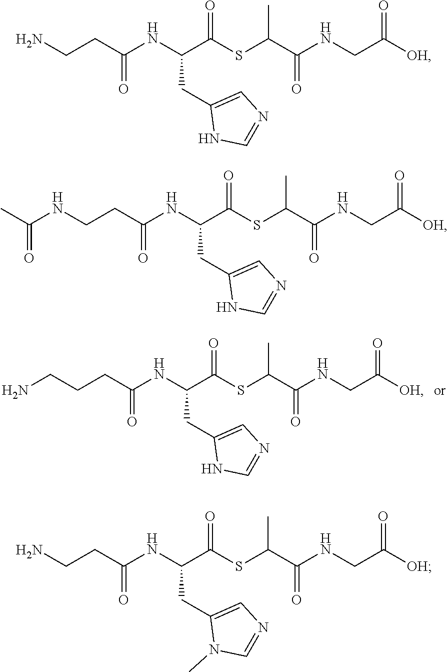

and when the condition, disease, or disorder is cystinuria, the compound is not stepronin or

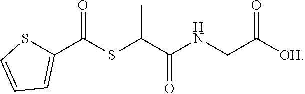 [SMILES: CC(SC(=O)c1cccs1)C(=O)NCC(=O)O]

In one embodiment, the condition, disease, or disorder is kidney disease. In one embodiment, the condition, disease, or disorder is a renal disease. In one embodiment, the condition, disease, or disorder is a bladder disease. In one embodiment, the condition, disease, or disorder is a urinary disease. In one embodiment, the condition, disease, or disorder is a ureteral disease. In one embodiment, the condition, disease, or disorder is kidney stones. In one embodiment, the condition, disease, or disorder is staghorn stones. In one embodiment, the condition, disease, or disorder is cystine stones. In one embodiment, the condition, disease, or disorder is calculi. In one embodiment, the condition, disease, or disorder is lithiasis. In one embodiment, the condition, disease, or disorder is nephrolithiasis. In one embodiment, the condition, disease, or disorder is urolithiasis. In one embodiment, the condition, disease, or disorder is urothiasis. In one embodiment, the condition, disease, or disorder is ureterolithiasis. In one embodiment, the condition, disease, or disorder is renolithiasis. In one embodiment, the condition, disease, or disorder comprises SLC3A1 gene mutations. In one embodiment, the condition, disease, or disorder comprises SLC7A9 gene mutations. In one embodiment, the condition, disease, or disorder comprises defects in the dibasic amino acid transporter. In one embodiment, the condition, disease, or disorder is cystinuria. In one embodiment, the condition, disease, or disorder is kidney stones, wherein the compound is not

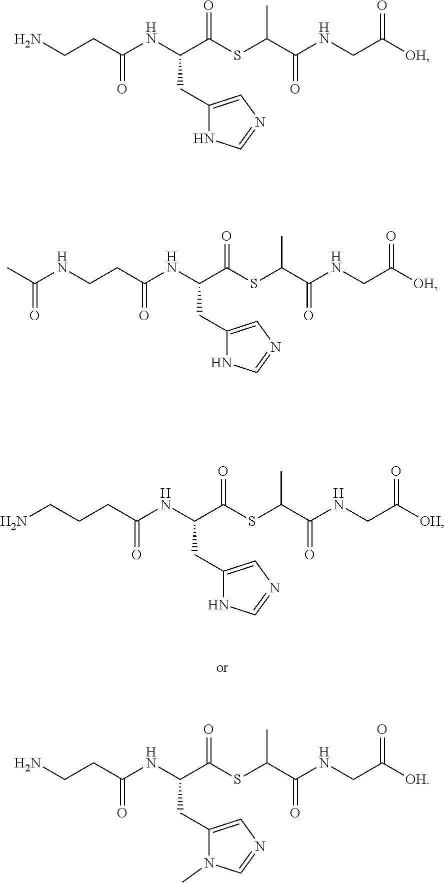 [SMILES: CC(SC(=O)[C@H](Cc1cnc[nH]1)NC(=O)CCN)C(=O)NCC(=O)O ; CC(=O)NCCC(=O)N[C@@H](Cc1cnc[nH]1)C(=O)SC(C)C(=O)NCC(=O)O ; CC(SC(=O)[C@H](Cc1cnc[nH]1)NC(=O)CCCN)C(=O)NCC(=O)O ; CC(SC(=O)[C@H](Cc1cncn1C)NC(=O)CCN)C(=O)NCC(=O)O]

In one embodiment, the condition, disease, or disorder is cystinuria, wherein the compound is not stepronin or

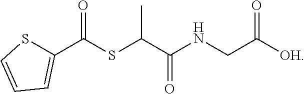 [SMILES: CC(SC(=O)c1cccs1)C(=O)NCC(=O)O]

In one embodiment, set forth herein is a method for treating cystinuria in a patient comprising administering to the patient a therapeutically effective amount of

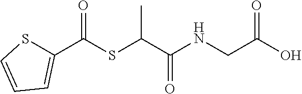 [SMILES: CC(SC(=O)c1cccs1)C(=O)NCC(=O)O]

having an ee greater than zero. In one embodiment, set forth herein is a method for treating a kidney disease, a renal disease, a bladder disease, a urinary disease, a ureteral disease, kidney stones, staghorn stones, cystine stones, calculi, lithiasis, nephrolithiasis, urolithiasis, urothiasis, ureterolithiasis, renolithiasis, SLC3A1 gene mutations, SLC7A9 gene mutations, defects in the dibasic amino acid transporter, and cystinuria in a patient comprising administering to the patient a therapeutically effective amount of

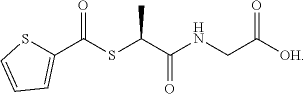 [SMILES: C[C@H](SC(=O)c1cccs1)C(=O)NCC(=O)O]

In one embodiment, set forth herein is a method for treating a kidney disease, a renal disease, a bladder disease, a urinary disease, a ureteral disease, kidney stones, staghorn stones, cystine stones, calculi, lithiasis, nephrolithiasis, urolithiasis, urothiasis, ureterolithiasis, renolithiasis, SLC3A1 gene mutations, SLC7A9 gene mutations, defects in the dibasic amino acid transporter, and cystinuria in a patient comprising administering to the patient a therapeutically effective amount of

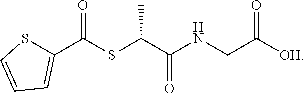 [SMILES: C[C@@H](SC(=O)c1cccs1)C(=O)NCC(=O)O]

In any of the embodiments in this paragraph, the compound is a pharmaceutically acceptable salt thereof.

### EXAMPLES

### General

In the examples below, unless otherwise stated, temperatures are given in degrees Celsius (° C.); operations were carried out at room or ambient temperature, “rt,” or “RT,” (typically a range of from about 18-25° C.; evaporation of solvent was carried out using a rotary evaporator under reduced pressure (typically 4.5-30 mm Hg) with a bath temperature of up to 60° C.; the course of reactions was typically followed by thin layer chromatography (TLC); melting points are uncorrected; products exhibited satisfactory ¹H NMR and/or microanalytical data; and the following conventional abbreviations are also used: L (liter(s)), mL (milliliters), mmol (millimoles), g (grams), mg (milligrams), min (minutes), and h (hours).

Unless otherwise specified, all solvents and reagents were purchased from suppliers and used without further purification. Reactions were conducted under a blanket of nitrogen unless otherwise stated. Compounds were visualized under a UV lamp (254 nm). ¹H NMR and ¹³C NMR spectra were recorded on a 300 MHz NMR instrument.

### Compounds

### Synthesis of Compound 1

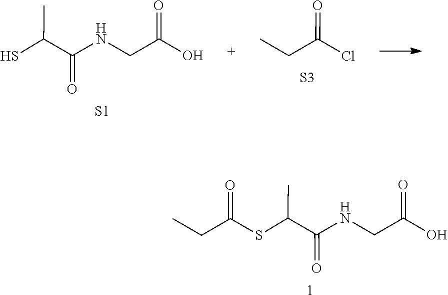

To a mixture of S1 (1.0 g, 6.1 mmol, 1.0 equiv) and TEA (1.2 g, 12.3 mmol, 2.0 equiv) in dioxane (5 mL) was added S3 (0.7 g, 7.4 mmol, 1.2 equiv) dropwise at 10° C. under nitrogen atmosphere. Then the mixture was stirred overnight at room temperature.

After the reaction was completed as indicated by TLC, the reaction was quenched with water (˜10 mL) and acidified to pH 2˜3 with 0.5 M HCl, followed by extraction with EA (10 mL×3). The combined organic layer was washed with water (10 mL) and dried over anhydrous sodium sulfate. After filtration and concentration, the crude product was obtained, which was purified by silica gel column chromatography (hexane:MTBE 1:10˜0:100 with 0.2% of AcOH) to provide ˜0.5 g of the product.

The product was further purified by slurry in hexane at room temperature overnight. After filtration, 0.5 g of the product was obtained. ¹H NMR was clean, but the Ellman's test was positive.

The product was then dissolved in MTBE and washed with CuSO₄ aqueous solution. The organic layer was dried over anhydrous sodium sulfate and concentrated to provide 290 mg of the product.

The product was then dissolved in acetone (40 mL). Cu(NO₃)₂ (200 mg) was added to the solution and the resulting mixture was stirred at room temperature overnight. Water (70 mL) was added to the mixture, followed by extraction with EA (70 mL, 50 mL, and 50 mL). The combined organic layer was washed with pure water (60 mL×3) and dried over anhydrous sodium sulfate. After filtration and concentration, 124 mg of the product was obtained .

The product was combined with another two batches (53 mg, and 53 mg) to provide 220 mg (8.2%) of compound 1. ¹H NMR (300 MHz, DMSO-d₆): δ 12.51 (br s, 1H), 8.44 (t, J=5.1 Hz, 1H), 4.17 (dd, J=14.1, 6.9 Hz, 1H), 3.73 (d, J=5.7 Hz, 2H), 2.59 (dd, J=14.7, 7.5 Hz, 2H), 1.36 (d, J=7.5 Hz, 3H), 1.09 (t, J=5.4 Hz, 3H).

### Synthesis of Compound 2

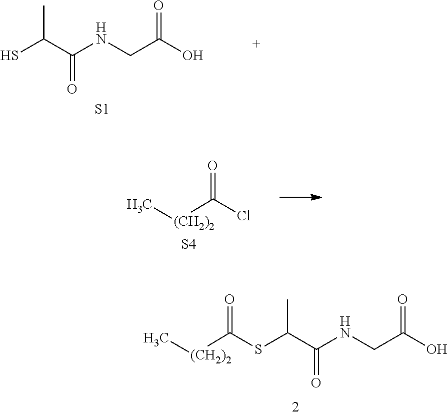

To a mixture of S1 (2.0 g, 12.2 mmol, 1.0 equiv) and TEA (2.5 g, 24.5 mmol, 2.0 equiv) in dry THF (10 mL) was added S4 (1.6 g, 14.7 mmol, 1.2 equiv) dropwise at 0° C. under nitrogen atmosphere. Then the mixture was stirred overnight at room temperature.

After the reaction was completed as indicated by TLC, the reaction was quenched with water (˜40 mL) and acidified to pH 2˜3 with 0.5 N HCl, followed by extraction with EA (30 mL×3). To the combined organic layer was added CuSO₄ aqueous solution and the mixture was stirred at room temperature for one hour. The organic layer was separated and treated with CuSO₄ aqueous solution (2×). The organic layer was separated and dried over anhydrous sodium sulfate. After filtration and concentration, the crude product was obtained, which was purified by silica gel column chromatography (hexane:MTBE 1:10˜0:100 with 0.2% of AcOH) to provide 795 mg of the product.

The product was dissolved with acetone (40 mL). Cu(NO₃)₂ (400 mg) was added to the solution and the resulting mixture was stirred at room temperature overnight. Water (70 mL) was added to the mixture, followed by extraction with EA (70 mL, 50 mL, and 50 mL). The combined organic layer was washed with pure water (60 mL×3) and dried over anhydrous sodium sulfate. After filtration and concentration, 690 mg of the product was obtained.

The product was further purified by slurry in hexane (2 mL) at room temperature. The isolated solid was slurried in hexane:MTBE (10:1, 5 mL) at room temperature overnight. After filtration, 361 mg (12.6%) of compound 3 was obtained. ¹H NMR (300 MHz, DMSO-d₆): δ 12.58 (br s, 1H), 8.44 (t, J=5.7 Hz, 1H), 4.17 (dd, J=14.1, 7.2 Hz, 1H), 3.73 (d, J=6.0 Hz, 2H), 2.58-2.50 (m, 2H), 1.58 (dd, J=14.7, 7.5 Hz, 2H), 1.40 (d, J=13.2 Hz, 3H), 0.86 (t, J=5.4 Hz, 3H).

### Synthesis of Compound 3

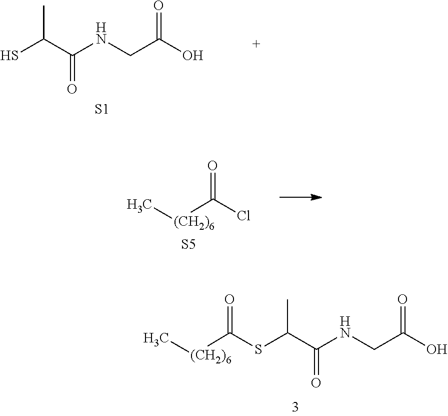

To a mixture of S1 (2.0 g, 12.2 mmol, 1.0 equiv) and TEA (2.5 g, 24.5 mmol, 2.0 equiv) in dry THF (10 mL) was added S5 (2.4 g, 14.7 mmol, 1.2 equiv) dropwise at 0° C. under nitrogen atmosphere. Then the mixture was stirred overnight at room temperature.

After the reaction was completed as indicated by TLC, the reaction was quenched with water (˜40 mL) and acidified to pH 2˜3 with 2 N HCl, followed by extraction with EA (30 mL×3). The combined organic layer was washed with water (30 mL×2) and dried over anhydrous sodium sulfate. After filtration and concentration, the crude product was obtained, which was purified by silica gel column chromatography (hexane:MTBE 1:10˜0:100 with 0.2% of AcOH) to provide 1.2 of the product.

The product was then dissolved in EA (50 mL) and washed with CuSO₄ aqueous solution (30 mL×3) and water (30 mL×2). The organic layer was separated and dried over anhydrous sodium sulfate. After filtration and concentration, 1.1 g of product was obtained.

The product was then dissolved in acetone (50 mL). Cu(NO₃)₂ (500 mg) was added to the solution and the resulting mixture was stirred at room temperature overnight. Water (70 mL) was added to the mixture, followed by extraction with EA (70 mL, 50 mL, and 50 mL). The combined organic layer was washed with pure water (60 mL×3) and dried over anhydrous sodium sulfate. After filtration and concentration, 1.3 g of the product was obtained.

The 1.3 g of product was further purified by slurry in hexane (2 mL) at room temperature overnight. After filtration, 1.0 g (28.2%) of compound 3 was obtained. ¹H NMR (300 MHz, DMSO-d6): δ 12.57 (br s, 1H), 8.44 (t, J=5.7 Hz, 1H), 4.17 (dd, J=14.1, 6.9 Hz, 1H), 3.73 (d, J=5.7 Hz, 2H), 2.59-2.50 (m, 2H), 1.55 (t, J=6.6 Hz, 2H), 1.36 (d, J=6.9 Hz, 3H), 1.24 (s, 8H), 0.85 (t, J=6.3 Hz, 3H).

### Synthesis of Compound 4

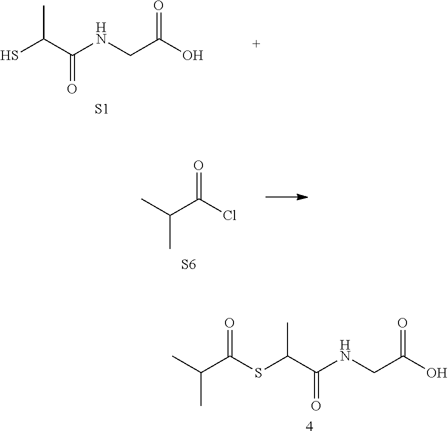

To a mixture of S1 (2.0 g, 12.2 mmol, 1.0 equiv) and TEA (2.5 g, 24.5 mmol, 2.0 equiv) in dry THF (10 mL) was added S6 (1.6 g, 14.7 mmol, 1.2 equiv) dropwise at 0° C. under nitrogen atmosphere. Then the mixture was stirred overnight at room temperature.

After the reaction was completed as indicated by TLC, the reaction was quenched with water (˜50 mL) and acidified to pH 2˜3 with 2 N HCl, followed by extraction with EA (30 mL×3). The combined organic layer was washed with water (30 mL×2) and dried over anhydrous sodium sulfate. After filtration and concentration, the crude product was obtained, which was purified by silica gel column chromatography (hexane:MTBE 1:10˜0:100 with 0.2% of AcOH) to provide 1.2 of the product.

The product was then dissolved in MTBE (50 mL) and washed with CuSO₄ aqueous solution (30 mL×3) and water (30 mL×2). The organic layer was separated and dried over anhydrous sodium sulfate. After filtration and concentration, 0.6 g of product was obtained.

The product was then dissolved in acetone (40 mL). Cu(NO₃)₂ (300 mg) was added to the solution and the resulting mixture was stirred at room temperature overnight. Water (70 mL) was added to the mixture, followed by extraction with EA (70 mL, 50 mL, and 50 mL). The combined organic layer was washed with pure water (60 mL×3) and dried over anhydrous sodium sulfate. After filtration and concentration, 475 mg (16.6%) of compound 4 was obtained. ¹H NMR (300 MHZ, DMSO-d6): δ0 12.57 (br s, 1H), 8.44 (t, J=5.7 Hz, 1H), 4.14 (dd, J=14.1, 6.9 Hz, 1H), 3.74 (d, J=6.0 Hz, 2H), 2.79-2.70 (m, 1H), 1.36 d, J=6.9 Hz, 3H), 1.18 (d, J=2.1 Hz, 6H).

### Synthesis of Compound 5

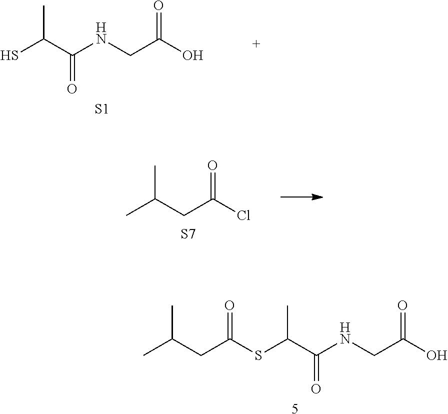

To a mixture of S1 (2.0 g, 12.2 mmol, 1.0 equiv) and TEA (2.5 g, 24.5 mmol, 2.0 equiv) in dry THF (10 mL) was added S7 (1.8 g, 14.7 mmol, 1.2 equiv) dropwise at 0° C. under nitrogen atmosphere. Then the mixture was stirred overnight at room temperature.

After the reaction was completed as indicated by TLC, the reaction was quenched with water (˜50 mL) and acidified to pH 2˜3 with 2 N HCl, followed by extraction with EA (30 mL×3). The combined organic layer was washed with water (30 mL×2) and dried over anhydrous sodium sulfate. After filtration and concentration, the crude product was obtained, which was purified by silica gel column chromatography (hexane:MTBE 1:10˜0:100 with 0.2% of AcOH) to provide 970 mg of the product.

The product was then dissolved in EA (50 mL) and washed with CuSO₄ aqueous solution (20 mL×3), citric acid aqueous solution (20 mL×3), and water (30 mL×2). The organic layer was separated and dried over anhydrous sodium sulfate. After filtration and concentration, 0.8 g of product was obtained.

The product was then dissolved in acetone (40 mL). Cu(NO₃)₂ (444 mg) was added to the solution and the resulting mixture was stirred at room temperature overnight. Water (70 mL) was added to the mixture, followed by extraction with EA (70 mL, 50 mL, and 50 mL). The combined organic layer was washed with pure water (60 mL×3) and dried over anhydrous sodium sulfate. After filtration and concentration, 726 mg of product was obtained. To the product was added Et₂O (2 mL) and the mixture was concentrated to dryness. To the residue was added hexane (4 mL) and the mixture was concentrated to dryness. 654 mg (21.6%) of compound 5 was obtained. ¹H NMR (300 MHZ, DMSO-d6): δ 12.58 (br s, 1H), 8.44 (t, J=5.7 Hz, 1H), 4.18 (dd, J=14.1, 6.9 Hz, 1H), 3.73 (d, J=6.0 Hz, 2H), 2.44 (d, J=6.9 Hz, 2H), 2.08-2.01 (m, 1H), 1.36 (d, J=6.9 Hz, 3H), 0.88 (d, J=6.9 Hz, 6H).

### Synthesis of Compound 6

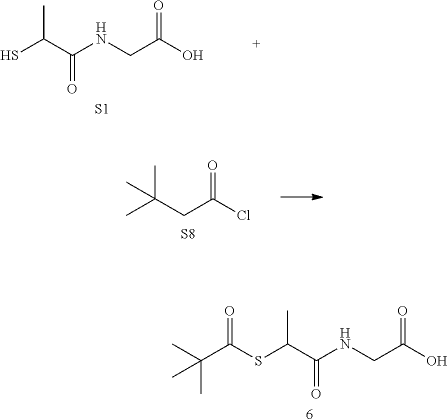

To a mixture of S1 (1.0 g, 6.1 mmol, 1.0 equiv) and TEA (1.2 g, 12.3 mmol, 2.0 equiv) in dioxane (5 mL) was added S8 (0.9 g, 7.3 mmol, 1.2 equiv) dropwise at 10° C. under nitrogen atmosphere. Then the mixture was stirred overnight at room temperature.

After the reaction was completed as indicated by TLC, the reaction was quenched with water (˜30 mL) and acidified to pH 2˜3 with 0.5 N HCl, followed by extraction with EA (30 mL×3). The combined organic layer was washed with water (30 mL×2) and dried over anhydrous sodium sulfate. After filtration and concentration, the crude product was obtained and combined with another batch 0.3 g) and purified by silica gel column chromatography (hexane:MTBE 1:10˜0:100 with 0.2% of AcOH) to provide 360 mg of the product.

The product was then dissolved in acetone (40 mL). Cu(NO₃)₂ (200 mg) was added to the solution and the resulting mixture was stirred at room temperature overnight. Water (70 mL) was added to the mixture, followed by extraction with EA (70 mL, 50 mL, and 50 mL). The combined organic layer was washed with pure water (60 mL×3) and dried over anhydrous sodium sulfate. After filtration and concentration, 200 mg (10.1%) of compound 6 was obtained. ¹H NMR (300 MHz, DMSO-d6): δ 12.57 (br s, 1H), 8.41 (t, J=5.4 Hz, 1H), 4.09 (dd, J=14.1, 7.2 Hz, 1H), 3.73 (d, J=5.4, 1.8 Hz, 2H), 1.35 (d, J=7.2 Hz, 3H), 1.17 (s, 9H).

### Synthesis of Compound 7

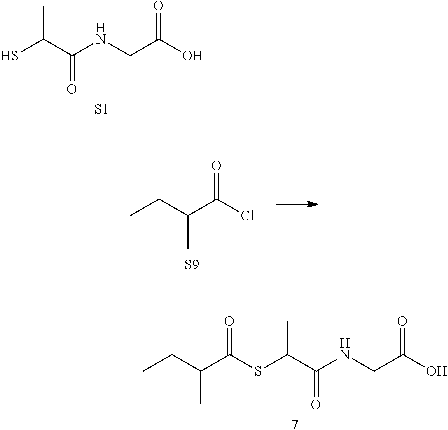

To a mixture of S1 (2.0 g, 6.1 mmol, 1.0 equiv) and TEA (2.5 g, 24.5 mmol, 2.0 equiv) in dry THF (10 mL) was added S9 (1.8 g, 14.7 mmol, 1.2 equiv) dropwise at 0° C. under nitrogen atmosphere. Then the mixture was stirred overnight at room temperature.

After the reaction was completed as indicated by TLC, the reaction was quenched with water (˜40 mL) and acidified to pH 2˜3 with 2 N HCl, followed by extraction with EA (30 mL×3). The combined organic layer was washed with CuSO₄ (30 mL×3) and dried over anhydrous sodium sulfate. After filtration and concentration, the crude product was obtained and purified by silica gel column chromatography (hexane:MTBE 1:10˜0:100 with 0.2% of AcOH) to provide 795 mg of the product.

The product was then dissolved in acetone (40 mL). Cu(NO₃)₂ (400 mg) was added to the solution and the resulting mixture was stirred at room temperature overnight. Water (70 mL) was added to the mixture, followed by extraction with EA (70 mL, 50 mL, and 50 mL). The combined organic layer was washed with pure water (60 mL×3) and dried over anhydrous sodium sulfate. After filtration and concentration, 690 mg of the product was obtained.

The product was then slurried overnight at room temperature in hexane:MTBE (10:1, 5 mL). After filtration, 361 mg (11.9%) of compound 7 was obtained. ¹H NMR (300 MHz, DMSO-d6): δ 12.57 (br s, 1H), 8.43 (d, J=4.8 Hz, 1H), 4.16 (dd, J=13.8, 6.9 Hz, 1H), 3.73 (d, J=6.0 Hz, 2H), 2.61 (dd, J=6.9, 3.6 Hz, 1H), 1.66-1.57 (m, 1H), 1.50-1.43 (m, 1H), 1.39 (d, J=7.8 Hz, 3H), 1.06 (d, J=6.9 Hz, 3H), 0.85 (t, J=7.5 Hz, 3H).

### Synthesis of Compound 8

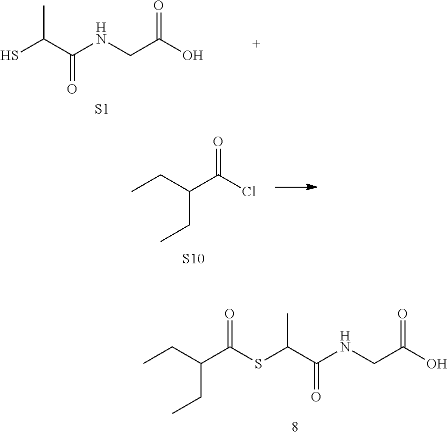

To a mixture of S1 (2.0 g, 6.1 mmol, 1.0 equiv) and TEA (2.5 g, 24.5 mmol, 2.0 equiv) in dry THF (10 mL) was added S10 (2.0 g, 14.7 mmol, 1.2 equiv) dropwise at 0° C. under nitrogen atmosphere. Then the mixture was stirred overnight at room temperature.

After the reaction was completed as indicated by TLC, the reaction was quenched with water (˜50 mL) and acidified to pH 2˜3 with 0.5 N HCl, followed by extraction with EA (30 mL×3). The combined organic layer was washed with water (30 mL) and dried over anhydrous sodium sulfate. After filtration and concentration, the crude product was obtained and purified by silica gel column chromatography (hexane:MTBE 1:10˜0:100 with 0.2% of AcOH) to provide 1.2 g of the product.

The product was then dissolved in acetone (40 mL). Cu(NO₃)₂ (444 mg) was added to the solution and the resulting mixture was stirred at room temperature overnight. Water (70 mL) was added to the mixture, followed by extraction with EA (70 mL, 50 mL, and 50 mL). The combined organic layer was washed with pure water (60 mL×3) and dried over anhydrous sodium sulfate. After filtration and concentration, 618 mg (19.1%) of compound 8 was obtained. ¹H NMR (300 MHz, DMSO-d6): δ 12.56 (br s, 1H), 8.44 (t, J=6.0 Hz, 1H), 4.20 (dd, J=14.1, 7.2 Hz, 1H), 3.73 (d, J=3.9 Hz, 2H), 2.46-2.40 (m, 1H), 1.61-1.52 (m, 4H), 1.36 (d, J=7.2 Hz, 3H), 0.84 (dd, J=7.8, 7.2 Hz, 6H).

### Synthesis of Compound 9

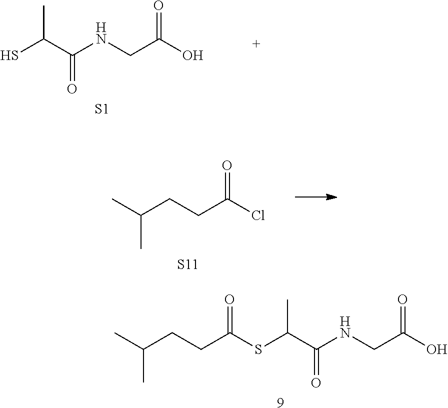

To a mixture of S1 (2.0 g, 6.1 mmol, 1.0 equiv) and TEA (2.5 g, 24.5 mmol, 2.0 equiv) in dry THF (10 mL) was added S11 (2.0 g, 14.7 mmol, 1.2 equiv) dropwise at 0° C. under nitrogen atmosphere. Then the mixture was stirred overnight at room temperature.

After the reaction was completed as indicated by TLC, the reaction was quenched with water (˜40 mL) and acidified to pH 2˜3 with 2 N HCl, followed by extraction with EA (30 mL×3). The combined organic layer was washed with water (30 mL) and dried over anhydrous sodium sulfate. After filtration and concentration, the crude product was obtained and purified by silica gel column chromatography (hexane:MTBE 1:10˜0:100 with 0.2% of AcOH) to provide 770 mg of the product.

The product was then dissolved in acetone (40 mL). Cu(NO₃)₂ (408 mg) was added to the solution and the resulting mixture was stirred at room temperature overnight. Water (70 mL) was added to the mixture, followed by extraction with EA (70 mL, 50 mL, and 50 mL). The combined organic layer was washed with pure water (60 mL×3) and dried over anhydrous sodium sulfate. After filtration and concentration, 686 mg of product was obtained and slurried in hexane (8 mL) at room temperature. After filtration, 541 mg (16.9%) of compound 9 was obtained. ¹H NMR (300 MHz, DMSO-d6): δ 12.55 (br s, 1H), 8.44 (t, J=5.7 Hz, 1H), 4.17 (dd, J=14.1, 7.2 Hz, 1H), 3.73 (d, J=6.0 Hz, 2H), 2.60-2.51 (m, 2H), 1.66-1.42 (m, 3H), 1.37 (d, J=7.2 Hz, 3H), 0.87 (d, J=7.8, 6H).

### Synthesis of Compound 10

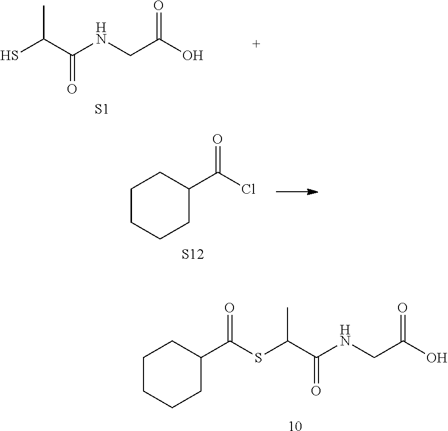

To a mixture of S1 (2.0 g, 6.1 mmol, 1.0 equiv) and TEA (2.5 g, 24.5 mmol, 2.0 equiv) in dry THF (10 mL) was added S12 (2.2 g, 14.7 mmol, 1.2 equiv) dropwise at 0° C. under nitrogen atmosphere. Then the mixture was stirred overnight at room temperature.

After the reaction was completed as indicated by TLC, the reaction was quenched with water (˜40 mL) and acidified to pH 2˜3 with 2 N HCl, followed by extraction with EA (30 mL×3). The combined organic layer was washed with water (30 mL) and dried over anhydrous sodium sulfate. After filtration and concentration, the crude product was obtained and purified by silica gel column chromatography (hexane:MTBE 1:10˜0:100 with 0.2% of AcOH) to provide 990 mg of the product.

The product was then dissolved in acetone (50 mL). Cu(NO₃)₂ (498 mg) was added to the solution and the resulting mixture was stirred at room temperature overnight. Water (70 mL) was added to the mixture, followed by extraction with EA (70 mL, 50 mL, and 50 mL). The combined organic layer was washed with pure water (60 mL×3) and dried over anhydrous sodium sulfate. After filtration and concentration, 796 mg of product was obtained and slurried in hexane (8 mL) at room temperature. After filtration, 683 mg (20.4%) of compound 10 was obtained. ¹H NMR (300 MHz, DMSO-d6): δ 12.58 (br s, 1H), 8.43 (t, J=6.0 Hz, 1H), 4.14 (dd, J=14.1, 6.9 Hz, 1H), 3.73 (d, J=6.0 Hz, 2H), 1.85-1.81 (m, 2H), 1.71-1.67 (m, 2H), 1.61-1.57 (m, 1H), 1.34 (d, J=6.9 Hz, 6H), 1.31 -16 (m, 3H).

### Synthesis of Compound 11

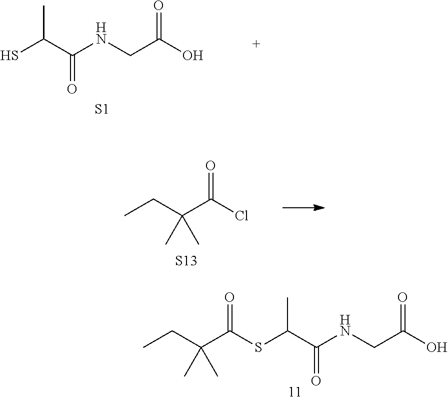

To a mixture of S1 (2.0 g, 6.1 mmol, 1.0 equiv) and TEA (2.5 g, 24.5 mmol, 2.0 equiv) in dry THF (10 mL) was added S13 (2.0 g, 14.7 mmol, 1.2 equiv) dropwise at 0° C. under nitrogen atmosphere. Then the mixture was stirred overnight at room temperature.

After the reaction was completed as indicated by TLC, the reaction was quenched with water (˜40 mL) and acidified to pH 2˜3 with 2 N HCl, followed by extraction with EA (30 mL×3). The combined organic layer was washed with water (30 mL) and dried over anhydrous sodium sulfate. After filtration and concentration, the crude product was obtained and purified by silica gel column chromatography (hexane:MTBE 1:10˜0:100 with 0.2% of AcOH) to provide 1.1 g of the product.

The product was then dissolved in acetone (50 mL). Cu(NO₃)₂ (500 mg) was added to the solution and the resulting mixture was stirred at room temperature overnight. Water (70 mL) was added to the mixture, followed by extraction with EA (70 mL, 50 mL, and 50 mL). The combined organic layer was washed with pure water (60 mL×3) and dried over anhydrous sodium sulfate. After filtration and concentration, 745 mg of product was obtained and slurried in hexane (8 mL) at room temperature. After filtration, 593 mg (18.5%) of compound 11 was obtained. ¹H NMR (300 MHz, DMSO-d6): δ 12.56 (br s, 1H), 8.41 (t, J=5.7 Hz, 1H), 4.11 (dd, J=14.1, 6.9 Hz, 1H), 3.74 (dd, J=5.7, 3.0 Hz, 2H), 1.55 (dd, J=15.0, 7.5 Hz, 2H), 1.35 (d, J=7.2 Hz, 3H), 1.13 (d, J=1.5 Hz, 6H), 0.79 (t, J=7.5 Hz, 3H).

### Synthesis of Compound 12

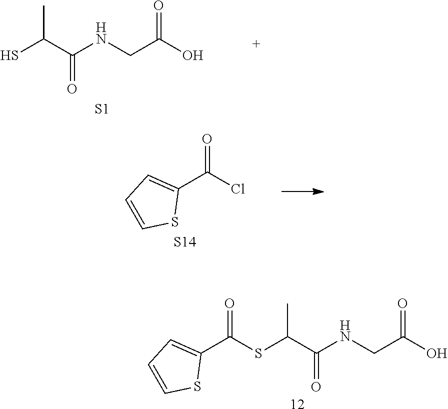

To a mixture of S1 (2.0 g, 6.1 mmol, 1.0 equiv) and TEA (2.5 g, 24.5 mmol, 2.0 equiv) in dry THF (10 mL) was added S14 (2.2 g, 14.7 mmol, 1.2 equiv) dropwise at 0° C. under nitrogen atmosphere. Then the mixture was stirred overnight at room temperature.

After the reaction was completed as indicated by TLC, the reaction was quenched with water (˜40 mL) and acidified to pH 2˜3 with 2 N HCl, followed by extraction with EA (30 mL×3). The combined organic layer was washed with water (30 mL) and dried over anhydrous sodium sulfate. After filtration and concentration, the crude product was obtained and purified by silica gel column chromatography (hexane:MTBE 1:10˜0:100 with 0.2% of AcOH) to provide 1.8 g of the product.

The product was then dissolved in EA (50 mL) and washed with CuSO₄ aqueous solution (20 mL×3). The organic layer was separated, washed with water (20 mL×2), and dried over sodium sulfate. After filtration and concentration, 1.6 g of the product was obtained.

The product was then dissolved in acetone (50 mL). Cu(NO₃)₂ (500 mg) was added to the solution and the resulting mixture was stirred at room temperature overnight. Water (70 mL) was added to the mixture, followed by extraction with EA (70 mL, 50 mL, and 50 mL). The combined organic layer was washed with pure water (60 mL×3) and dried over anhydrous sodium sulfate. After filtration and concentration, 947 mg of product was obtained and slurried overnight in MTBE (4 mL) at room temperature. After filtration, 637 mg (19.0%) of compound 12 was obtained. ¹H NMR (300 MHz, DMSO-d₆): δ 12.61 (br s, 1H), 8.57 (t, J=5.7 Hz, 1H), 8.07 (dd, J=4.8, 0.9 Hz, 1H), 7.91 (dd, J=3.9, 1.2 Hz, 1H), 7.26 (dd, J=5.1, 3,9 Hz, 1H), 4.40 (dd, J=14.1, 6.9 Hz, 1H), 3.77 (d, J=5.7 Hz, 2H), 1.47 (d, J=6.9 Hz, 3H).

### Synthesis of Compound 13

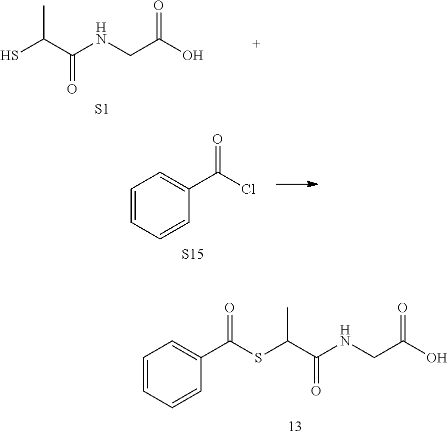

To a mixture of S1 (2.0 g, 6.1 mmol, 1.0 equiv) and TEA (2.5 g, 24.5 mmol, 2.0 equiv) in dry THF (10 mL) was added S15 (2.1 g, 14.7 mmol, 1.2 equiv) dropwise at 0° C. under nitrogen atmosphere. Then the mixture was stirred overnight at room temperature.

After the reaction was completed as indicated by TLC, the reaction was quenched with water (˜40 mL) and acidified to pH 2˜3 with 2 N HCl, followed by extraction with EA (30 mL×3). The combined organic layer was washed with water (30 mL) and dried over anhydrous sodium sulfate. After filtration and concentration, the crude product was obtained and purified by silica gel column chromatography (hexane: MTBE 1:10˜0:100 with 0.2% of AcOH) to provide 1.1 g of the product.

The product was then dissolved in acetone (50 mL). Cu(NO₃)₂ (500 mg) was added to the solution and the resulting mixture was stirred at room temperature overnight. Water (70 mL) was added to the mixture, followed by extraction with EA (70 mL, 50 mL, and 50 mL). The combined organic layer was washed with pure water (60 mL×3) and dried over anhydrous sodium sulfate. After filtration and concentration, 876 mg (26.8%) of compound 13 was obtained. ¹H NMR (300 MHz, DMSO-d6): δ 12.60 (br s, 1H), 8.57 (t, J=5.7 Hz, 1H), 7.91 (d, J=7.2 Hz, 2H), 7.71 (t, J=7.5 Hz, 1H), 7.57 (dd, J=7.8 Hz, 2H), 4.40 (dd, J=13.8, 6.9 Hz, 1H), 4.03 (d, J=6.9 Hz, 2H), 1.50 (dd, J=14.7, 6.9 Hz, 3H).

### Synthesis of Compound 14

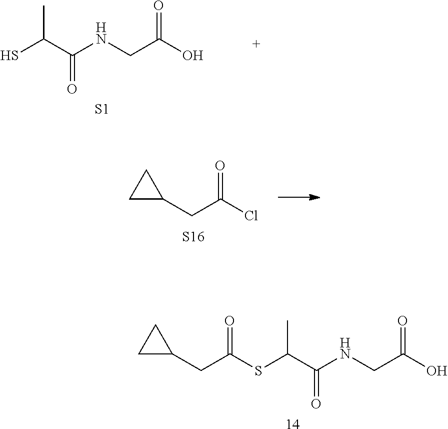

To a stirred solution of compound S1 (0.5 g, 3.067 mmol, 1.0 equiv) in toluene (5 mL) was added NaOH (0.306 g, 7.667 mmol, 2.5 equiv) dissolved in water (5 mL) at 0° C. The reaction was then stirred at 0° C. for 30 min. To this solution was added compound S16 (0.43 g, 3.68 mmol, 1.2 equiv) at 0° C. The reaction mixture was then stirred at room temperature for 3 h. After completion of the reaction (as monitored by TLC), the reaction mixture was acidified with 2 N HCl and extracted with EtOAc (20 mL). The combined organic layers were washed with water (10 mL) followed by brine (5 mL), dried over anhydrous Na₂SO₄, and concentrated under reduced pressure. The crude compound was purified by Combi-Flash chromatography (eluting with 1-5% MeOH in DCM) to afford compound 14 (0.5 g, 66% yield) as an off-white solid. ¹H NMR (400 MHZ, DMSO-d₆) δ 12.57 (br s, 1H), 8.43 (br s, 1H), 4.18 (q, J=6.8 Hz, 1H), 3.73 (d, J=4.0 Hz, 2H), 2.48-2.46 (m, 2H,), 1.37 (br d, J=6.8 Hz, 3H), 0.96 (br s, 1H), 0.51 (br d, J=7.3 Hz, 2H), 0.19 (br d, J=3.9 Hz, 2H). HPLC purity: 99.84%; LCMS calculated for C₁₀H₁₅NO₄S: 245.29; Observed: 246.15 (M+1)⁺.

### Synthesis of Compound 15

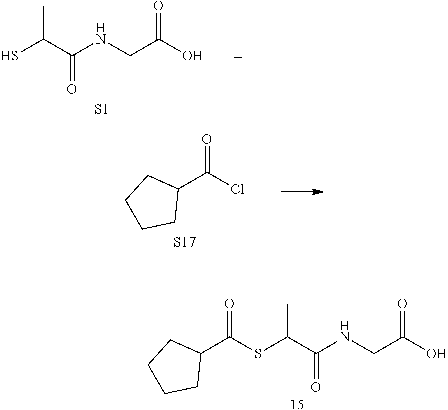

To a stirred solution of compound S1 (0.5 g, 3.067 mmol, 1.0 equiv) in toluene (5 mL) was added NaOH (0.306 g, 7.667 mmol, 2.5 equiv) dissolved in water (5 mL) at 0° C. The reaction was then stirred at 0° C. for 30 min. To this solution was added compound S17 (0.48 g, 3.68 mmol, 1.2 equiv) at 0° C. The reaction mixture was then stirred at room temperature for 3 h. After completion of the reaction (as monitored by TLC), the reaction mixture was acidified with 2 N HCl and extracted with EtOAc (20 mL). The combined organic layers were washed with water (10 mL) followed by brine (5 mL), dried over anhydrous Na₂SO₄, and concentrated under reduced pressure. The crude compound was purified by Combi-Flash chromatography (eluting with 1-5% MeOH in DCM) to afford compound 15 (0.5 g, 63% yield) as an off-white solid. ¹H NMR (400 MHZ, DMSO-d₆) δ 12.57 (br s, 1H), 8.37 (t, J=5.6 Hz, 1H), 4.15 (q, J=6.8 Hz, 1H), 3.72 (q, J=1.6, 2H), 3.00 (quin, J=7.7 Hz, 1H), 1.86-1.82 (m, 2H), 1.76-1.66 (m, 2H), 1.66-1.49 (m, 4H), 1.36 (d, J=7.3 Hz, 3H). HPLC purity: 99.56%; LCMS calculated for C₁₁H₁₇NO₄S: 259.52: Observed: 260.17 (M+1)⁺.

### Synthesis of Compound 16

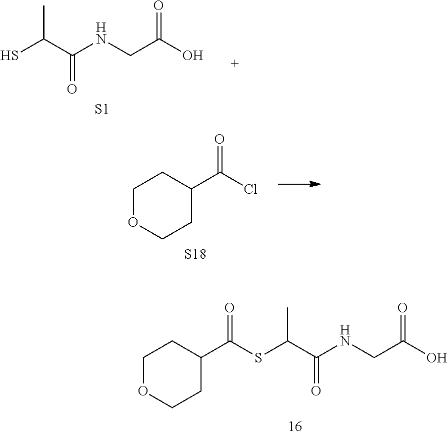

To a stirred solution of compound S1 (0.4 g, 0.44 mmol, 1.02 equiv) in toluene (4 mL) was added NaOH (0.22 g, 5.656 mmol, 2.1 equiv) dissolved in water (5 mL) at 0° C. The reaction was then stirred at 0° C. for 30 min. To this solution was added compound S18 (0.48 g, 3.68 mmol, 1.2 equiv) dissolved in toluene (2 mL) at 0° C. The reaction mixture was then stirred at room temperature for 2 h. After completion of the reaction (as monitored by TLC), the reaction mixture was acidified with 2 N HCl and extracted with EtOAc (20 mL). The combined organic layers were washed with water (10 mL) followed by brine (5 mL), dried over anhydrous Na₂SO₄, and concentrated under reduced pressure. The crude compound was purified by Combi-Flash chromatography (eluting with 0-5% MeOH in DCM) to afford compound 16 (0.25 g, 33.7% yield) as an off-white solid. ¹H NMR (400 MHz, DMSO-d₆) δ 12.34 (br s, 1H), 8.43 (t, J=5.6 Hz, 1H), 4.16 (q, J=6.8 Hz, 1H), 3.85-3.81 (m, 2H), 3.37-3.3 (m, 2H), 2.83-2.78 (m, 1H), 1.76-1.72 (m, 2H), 1.62-1.52 (m, 2H), 1.34 (d, J=7.1 Hz, 3H). HPLC purity: 99.34%; LCMS calculated for C₁₁H₁₇NO₅S: 275.32; Observed: 275.85 (M+1)⁺.

### Formation of Tiopronin From Incubation of the Prodrugs in Human Liver Microsomes

Method: Prodrugs were incubated with human liver microsomes at a protein concentration of 2 mg/mL prepared in potassium phosphate buffer pH 7.4 in a 96-well format in duplicate. The liver microsomal mixture was preincubated for 5 min at 37° C. in a Heidolpho plate shaker. The reaction was initiated by addition of test compounds. Final concentration of prodrugs in the assay was 10 μM. Samples were collected at 0, 30, 60, 90, and 120 min and quenched with acetonitrile containing an internal standard. Samples were vortex mixed and centrifuged at 4,000 RPM for 10 min. The clear supernatants were transferred to fresh 96-well plate and analyzed by Fit for purpose LC-MS/MS method to quantify both parent prodrug and the active drug tiopronin in the test samples. The table below shows the % Tiopronin measured after 60 min incubation of the prodrugs in human liver microsomes.

| | % Tiopronin |
|---|---|
| Compound | Measured After |
| Number | 60 min Incubation |
| 5 | 14.4 |
| 7 | 20.3 |
| 10 | 100 |
| 11 | 5.4 |
| 12 (Stepronin) | 68.5 |
| 13 | 25.3 |
| 14 | 50.1 |
| 15 | 88.4 |
| 16 | 9.9 |

The data shows that the tested prodrugs produce the active drug, Tiopronin, in different amounts allowing one to select the prodrug that produces the desired amount of Tiopronin for the treatment of patients.

What is claimed is:

1. A compound of the following formula 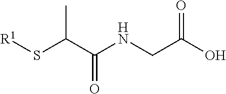
wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl;
wherein alkyl, alkenyl, alkynyl, cycloalkyl, aryl, and heteroaryl are optionally substituted and/or comprise one or more stereogenic centers;
wherein R¹ is not methyl, CH₃(CH₂)₁₆—, CH₃(CH₂)₁₄—, CH₃(CH₂)₁₂—, or CH₃(CH₂)₁₀—, 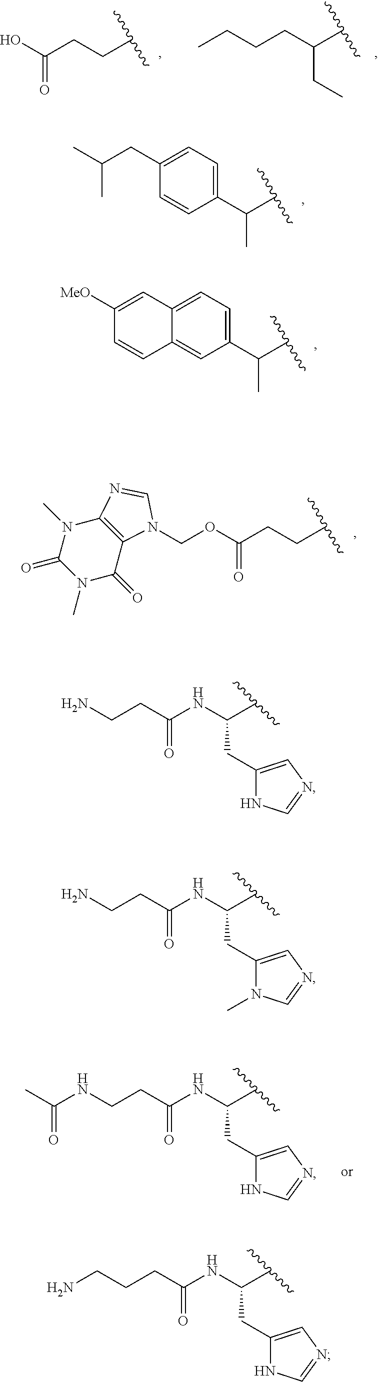
wherein R¹ is not phenyl, 2-acetoxyphenyl, 4-nitrophenyl, or 3,4,5-trimethoxyphenyl;
wherein R¹ is not 3-pyridyl;
wherein R¹ is not 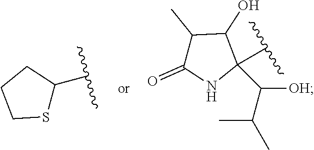
wherein R¹ is not 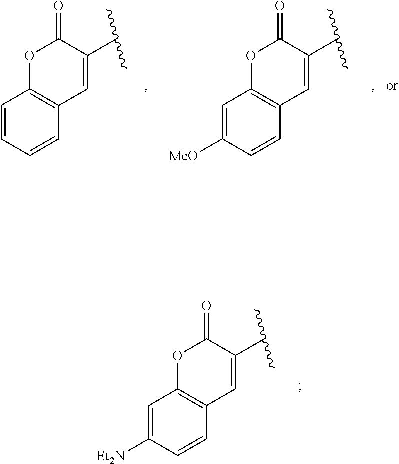
and
wherein when R¹ is 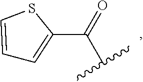 [SMILES: *C(=O)c1cccs1]
then the compound has an ee greater than zero; or
a pharmaceutically acceptable salt thereof.

2. The compound of claim 1, wherein the compound is 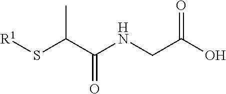
wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl;
wherein alkyl, alkenyl, alkynyl, cycloalkyl, aryl, and heteroaryl are optionally substituted and/or comprise one or more nonracemic stereogenic centers;
wherein R¹ is not 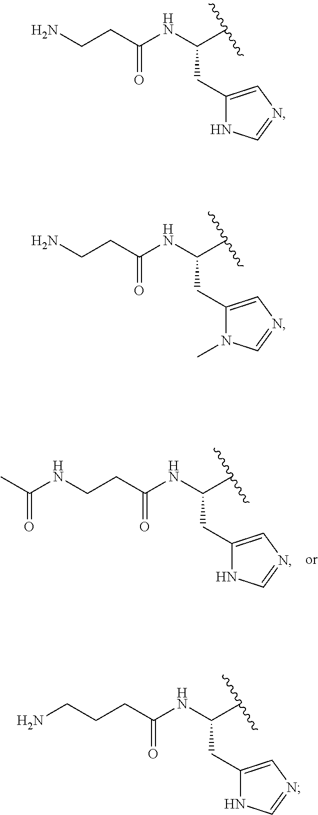 [SMILES: *[C@H](Cc1cnc[nH]1)NC(=O)CCN ; *[C@H](Cc1cncn1C)NC(=O)CCN ; *[C@H](Cc1cnc[nH]1)NC(=O)CCNC(C)=O ; *[C@H](Cc1cnc[nH]1)NC(=O)CCCN]
or
a stereoisomer thereof or a pharmaceutically acceptable salt thereof.

3. The compound of claim 1, wherein the compound is 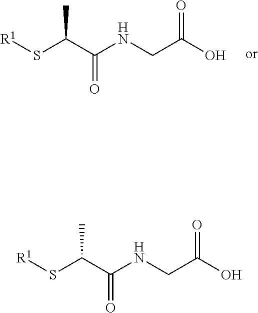
wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl;
wherein alkyl, alkenyl, alkynyl, cycloalkyl, aryl, and heteroaryl are optionally substituted and/or comprise one or more stereogenic centers; or
a pharmaceutically acceptable salt thereof.

4. The compound of claim 1, wherein the compound is 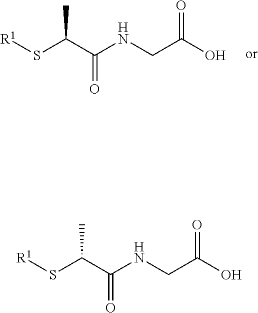
wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl;
wherein alkyl, alkenyl, alkynyl, cycloalkyl, aryl, and heteroaryl are optionally substituted and/or comprise one or more nonracemic stereogenic centers; or
a pharmaceutically acceptable salt thereof.

5. The compound of claim 1, wherein R¹ is —(C═O)aryl;
wherein aryl is optionally substituted; and
wherein aryl is not phenyl, 2-acetoxyphenyl, 4-nitrophenyl, or 3,4,5-trimethoxyphenyl; or
a pharmaceutically acceptable salt thereof.

6. The compound of claim 1, wherein R¹ is —(C═O)heteroaryl;
wherein heteroaryl is optionally substituted;
wherein heteroaryl is not 3-pyridyl; and
wherein when R¹ is 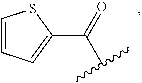 [SMILES: *C(=O)c1cccs1]
then the compound has an ee greater than zero; or
a pharmaceutically acceptable salt thereof.

7. The compound of any preceding claim, wherein when alkyl, alkenyl, alkynyl, cycloalkyl, aryl, or heteroaryl is substituted, then the substituents are selected from the group consisting of alkyl, alkenyl, alkynyl, cycloalkyl, aryl, heteroaryl, amino, halo, cyano, nitro, hydroxy, acyl, amido, —C(O)—, —C(S)—, haloalkyl, and azido.

8. The compound of claim 6, wherein the compound is 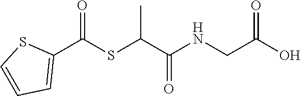 [SMILES: CC(SC(=O)c1cccs1)C(=O)NCC(=O)O]
having an ee greater than zero; or
a pharmaceutically acceptable salt thereof.

9. The compound of claim 6, wherein the compound is 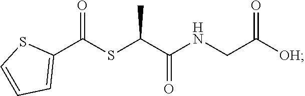 [SMILES: C[C@H](SC(=O)c1cccs1)C(=O)NCC(=O)O]
or
a pharmaceutically acceptable salt thereof.

10. The compound of claim 6, wherein the compound is 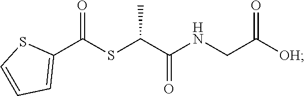 [SMILES: C[C@@H](SC(=O)c1cccs1)C(=O)NCC(=O)O]
or
a pharmaceutically acceptable salt thereof.

11. A compound of the following formula 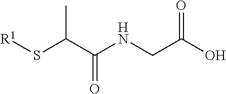
wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl;
wherein alkyl, alkenyl, alkynyl, cycloalkyl, aryl, and heteroaryl are optionally substituted;
wherein the compound has an ee greater than zero; or
a pharmaceutically acceptable salt thereof.

12. The compound of claim 11, wherein alkyl, alkenyl, alkynyl, cycloalkyl, aryl, and heteroaryl are optionally substituted and/or comprise one or more stereogenic centers; and
wherein the compound has an ee or de greater than zero.

13. The compound of claim 11, wherein alkyl is methyl, CH₃(CH₂)₁₆—, CH₃(CH₂)₁₄—, CH₃(CH₂)₁₂—, CH₃(CH₂)₁₀—, 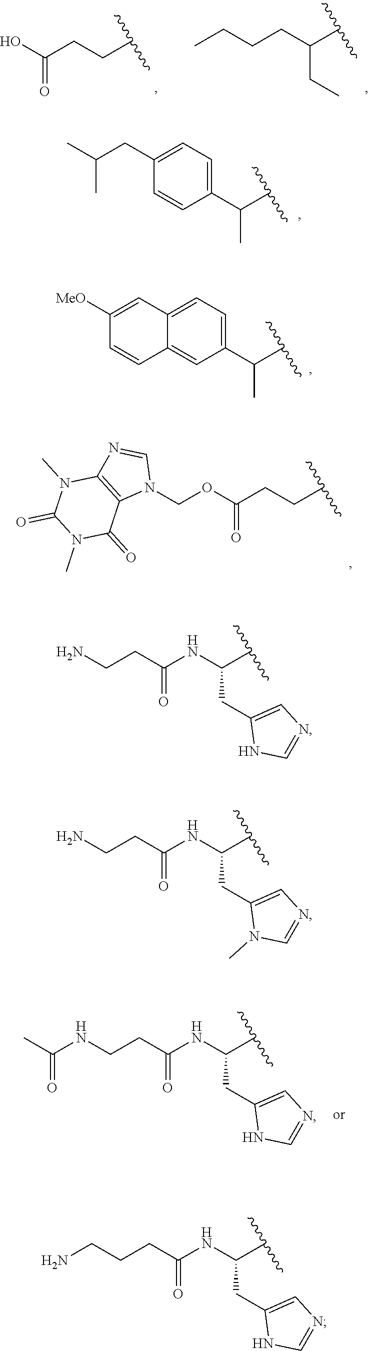
or
wherein heterocycloalkyl is 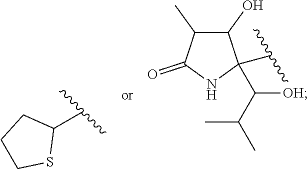 [SMILES: *C1CCCS1 ; *C1(C(O)C(C)C)NC(=O)C(C)C1O]
and
wherein the compound has an ee or de greater than zero; or
a pharmaceutically acceptable salt thereof.

14. The compound of any one of claims 11-13, wherein when alkyl, alkenyl, alkynyl, cycloalkyl, aryl, or heteroaryl is substituted, then the substituents are selected from the group consisting of alkyl, alkenyl, alkynyl, cycloalkyl, aryl, heteroaryl, amino, halo, cyano, nitro, hydroxy, acyl, amido, —C(O)—, —C(S)—, haloalkyl, and azido.

15. The compound of claim 13, wherein alkyl is methyl and the compound has an ee greater than zero; or a pharmaceutically acceptable salt thereof.

16. The compound of claim 13, wherein alkyl is CH₃(CH₂)₁₆— and the compound has an ee greater than zero; or a pharmaceutically acceptable salt thereof.

17. The compound of claim 13, wherein alkyl is CH₃(CH₂)₁₄— and the compound has an ee greater than zero; or a pharmaceutically acceptable salt thereof.

18. The compound of claim 13, wherein alkyl is CH₃(CH₂)₁₂— and the compound has an ee greater than zero; or a pharmaceutically acceptable salt thereof.

19. The compound of claim 13, wherein alkyl is CH₃(CH₂)₁₀— and the compound has an ee greater than zero; or a pharmaceutically acceptable salt thereof.

20. The compound of claim 13, wherein alkyl is 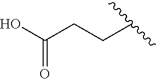 [SMILES: *CCC(=O)O]
and the compound has an ee greater than zero; or a pharmaceutically acceptable salt thereof.

21. The compound of claim 13, wherein alkyl is 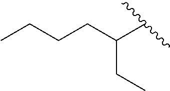 [SMILES: *C(CC)CCCC]
and the compound has an ee or de greater than zero; or a pharmaceutically acceptable salt thereof.

22. The compound of claim 13, wherein alkyl is 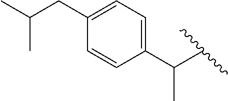 [SMILES: *C(C)c1ccc(CC(C)C)cc1]
and the compound has an ee or de greater than zero; or a pharmaceutically acceptable salt thereof.

23. The compound of claim 13, wherein alkyl is 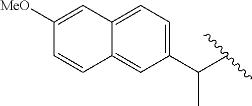 [SMILES: *C(C)c1ccc2cc(OC)ccc2c1]
and the compound has an ee or de greater than zero; or a pharmaceutically acceptable salt thereof.

24. The compound of claim 13, wherein alkyl is 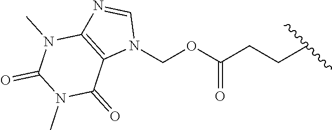 [SMILES: *CCC(=O)OCn1cnc2c1c(=O)n(C)c(=O)n2C]
and the compound has an ee greater than zero; or a pharmaceutically acceptable salt thereof.

25. The compound of claim 13, wherein alkyl is 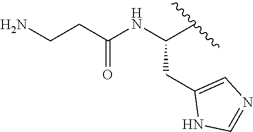 [SMILES: *[C@H](Cc1cnc[nH]1)NC(=O)CCN]
and the compound has an ee or de greater than zero; or a pharmaceutically acceptable salt thereof.

26. The compound of claim 13, wherein alkyl is 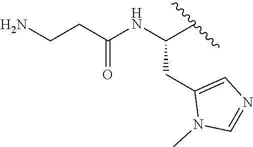 [SMILES: *[C@H](Cc1cncn1C)NC(=O)CCN]
and the compound has an ee or de greater than zero; or a pharmaceutically acceptable salt thereof.

27. The compound of claim 13, wherein alkyl is 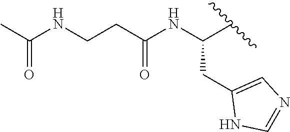 [SMILES: *[C@H](Cc1cnc[nH]1)NC(=O)CCNC(C)=O]
and the compound has an ee or de greater than zero; or a pharmaceutically acceptable salt thereof.

28. The compound of claim 13, wherein alkyl is 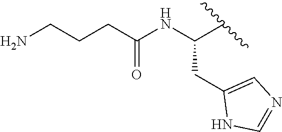 [SMILES: *[C@H](Cc1cnc[nH]1)NC(=O)CCCN]
and the compound has an ee or de greater than zero; or a pharmaceutically acceptable salt thereof.

29. The compound of claim 13, wherein heterocycloalkyl is 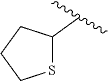 [SMILES: *C1CCCS1]
and the compound has an ee or de greater than zero; or a pharmaceutically acceptable salt thereof.

30. The compound of claim 13, wherein heterocycloalkyl is 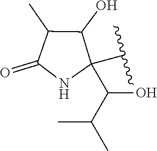 [SMILES: *C1(C(O)C(C)C)NC(=O)C(C)C1O]
and the compound has an ee or de greater than zero; or a pharmaceutically acceptable salt thereof.

31. A pharmaceutical composition comprising the compound of any one of claims 1-30, or a pharmaceutically acceptable salt thereof and a pharmaceutically acceptable carrier.

32. A method for treating a condition, disease, or disorder in a patient comprising administering to the patient a therapeutically effective amount of a compound of the following formula 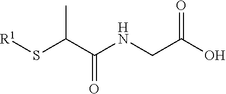
wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl;
wherein alkyl, alkenyl, alkynyl, cycloalkyl, aryl, and heteroaryl are optionally substituted and/or comprise one or more stereogenic centers; and
wherein when R¹ is 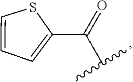
then the compound has an ee greater than zero; or
a pharmaceutically acceptable salt thereof.

33. The method of claim 32, wherein the compound is 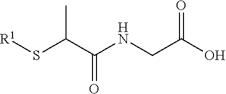
wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl;
wherein alkyl, alkenyl, alkynyl, cycloalkyl, aryl, and heteroaryl are optionally substituted and/or comprise one or more nonracemic stereogenic centers; or
a stereoisomer thereof or a pharmaceutically acceptable salt thereof.

34. The method of claim 32, wherein the compound is 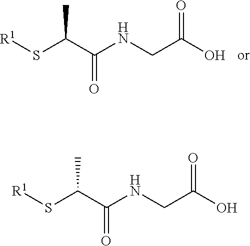
wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl;
wherein alkyl, alkenyl, alkynyl, cycloalkyl, aryl, and heteroaryl are optionally substituted and/or comprise one or more stereogenic centers; or
a pharmaceutically acceptable salt thereof.

35. The method of claim 32, wherein the compound is 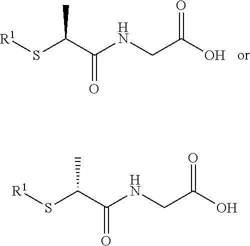
wherein R¹ is —(C═O)alkyl, —(C═O)alkenyl, —(C═O)alkynyl, —(C═O)cycloalkyl, —(C═O)alkoxyalkyl, —(C═O)heterocyclyl, —(C═O)heterocycloalkyl, —(C═O)aryl, —(C═O)arylalkyl, or —(C═O)heteroaryl;
wherein alkyl, alkenyl, alkynyl, cycloalkyl, aryl, and heteroaryl are optionally substituted and/or comprise one or more nonracemic stereogenic centers; or
a pharmaceutically acceptable salt thereof.

36. The method of claim 32, wherein R¹ is —(C═O)aryl;
wherein aryl is optionally substituted; or
a pharmaceutically acceptable salt thereof.

37. The method of claim 32, wherein R¹ is —(C═O)heteroaryl;
wherein heteroaryl is optionally substituted; or
a pharmaceutically acceptable salt thereof.

38. The compound of any one of claims 32-37, wherein when alkyl, alkenyl, alkynyl, cycloalkyl, aryl, or heteroaryl is substituted, then the substituents are selected from the group consisting of alkyl, alkenyl, alkynyl, cycloalkyl, aryl, heteroaryl, amino, halo, cyano, nitro, hydroxy, acyl, amido, —C(O)—, —C(S)—, haloalkyl, and azido.

39. The method of claim 32, wherein the compound is 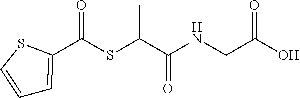 [SMILES: CC(SC(=O)c1cccs1)C(=O)NCC(=O)O]
having an ee greater than zero; or a pharmaceutically acceptable salt thereof.

40. The method of claim 39, wherein the compound is 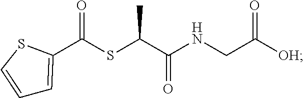 [SMILES: C[C@H](SC(=O)c1cccs1)C(=O)NCC(=O)O]
or a pharmaceutically acceptable salt thereof.

41. The method of claim 39, wherein the compound is 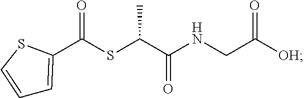 [SMILES: C[C@@H](SC(=O)c1cccs1)C(=O)NCC(=O)O]
or a pharmaceutically acceptable salt thereof.

42. The method of any one of claims 32-41, wherein the condition, disease, or disorder is selected from the group consisting of a kidney disease, a renal disease, a bladder disease, a urinary disease, a ureteral disease, kidney stones, staghorn stones, cystine stones, calculi, lithiasis, nephrolithiasis, urolithiasis, urothiasis, ureterolithiasis, renolithiasis, SLC3A1 gene mutations, SLC7A9 gene mutations, defects in the dibasic amino acid transporter, and cystinuria;
wherein when the condition, disease, or disorder is kidney stones, the compound is not 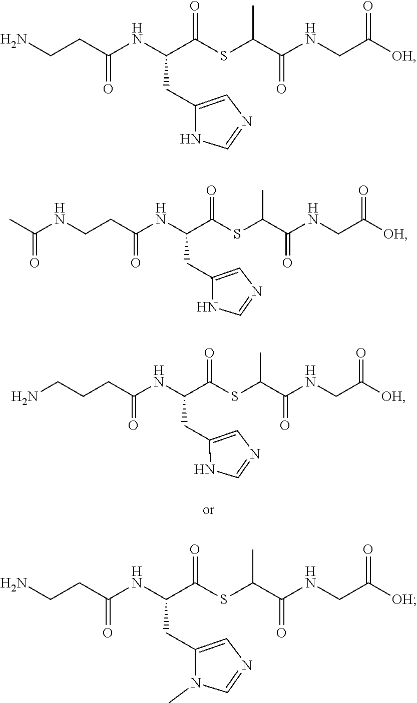 [SMILES: CC(SC(=O)[C@H](Cc1cnc[nH]1)NC(=O)CCN)C(=O)NCC(=O)O ; CC(=O)NCCC(=O)N[C@@H](Cc1cnc[nH]1)C(=O)SC(C)C(=O)NCC(=O)O ; CC(SC(=O)[C@H](Cc1cnc[nH]1)NC(=O)CCCN)C(=O)NCC(=O)O ; CC(SC(=O)[C@H](Cc1cncn1C)NC(=O)CCN)C(=O)NCC(=O)O]
and when the condition, disease, or disorder is cystinuria, the compound is not stepronin or

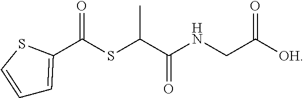 [SMILES: CC(SC(=O)c1cccs1)C(=O)NCC(=O)O]

43. The method of claim 42, wherein the condition, disease, or disorder is cystinuria.

44. A method for treating a kidney disease, a renal disease, a bladder disease, a urinary disease, a ureteral disease, kidney stones, staghorn stones, cystine stones, calculi, lithiasis, nephrolithiasis, urolithiasis, urothiasis, ureterolithiasis, renolithiasis, SLC3A1 gene mutations, SLC7A9 gene mutations, defects in the dibasic amino acid transporter, and cystinuria in a patient comprising administering to the patient a therapeutically effective amount of a compound of the following formula having an ee greater than zero 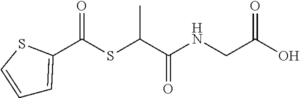 [SMILES: CC(SC(=O)c1cccs1)C(=O)NCC(=O)O]
or a pharmaceutically acceptable salt thereof.

45. The method of claim 44, wherein the method is for treating cystinuria.

46. The method of claim 44, wherein the compound is 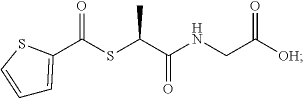 [SMILES: C[C@H](SC(=O)c1cccs1)C(=O)NCC(=O)O]
or a pharmaceutically acceptable salt thereof.

47. The method of claim 44, wherein the compound is 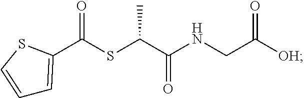 [SMILES: C[C@@H](SC(=O)c1cccs1)C(=O)NCC(=O)O]
or a pharmaceutically acceptable salt thereof.
